(19) United States
(12) Patent Application Publication
(10) Pub. No.: US 2011/0196141 A1
(43) Pub. Date: Aug. 11, 2011

(54) LOCKED AND UNLOCKED 2'-O PHOSPHORAMIDITE NUCLEOSIDES, PROCESS OF PREPARATION THEREOF AND OLIGOMERS COMPRISING THE NUCLEOSIDES

(75) Inventors: Anil Kumar VAIJAYANTI, Pune (IN); Anita Dinkar GUNJAI, Pune (IN); Moneesha D'COSTA, Pune (IN); Namrata Diliprao ERANDE, Pune (IN); Venubabu KOTIKAM, Pune (IN)

(73) Assignee: COUNCIL OF SCIENTIFIC & INDUSTRIAL RESEARCH, New Delhi (IN)

(21) Appl. No.: 12/876,494

(22) Filed: Sep. 7, 2010

(30) Foreign Application Priority Data
Sep. 7, 2009 (IN) 1837DEL/2009

(51) Int. Cl. C07H 21/02; C07H 19/20; C07H 19/10; C07H 21/04

(52) U.S. Cl. 536/23.1; 536/26.7; 536/26.8

(57) ABSTRACT

The present invention relates to 2′-O-phosphoramidite of locked nucleoside and unlocked nucleoside, their synthesis and 2′-5′-linked oligomers oligomers comprising the nucleosides to delineate the structural requirements of 2′-5′ RNA/DNA: 3′-5′ RNA duplexes and also for use in antisense applications.

This Application claims the priority of Indian Patent Application No. 1837DEL/2009, filed Sep. 7, 2009, which is incorporated herein by reference.

### FIELD OF THE INVENTION

The present invention relates to 2′-O-phosphoramidite locked nucleosides, and 2′-O-phosphoramidite unlocked nucleosides, their synthesis and 2′-5′-linked oligomers comprising the nucleosides to delineate the structural requirements of 2′-5′ RNA/DNA: 3′-5′ RNA duplexes and also their use in antisense applications.

### BACKGROUND AND PRIOR ART

Locked Nucleic Acid (LNA) was first described by Wengel and co-workers in 1998, as a novel class of conformationally restricted oligonucleotide analogues. LNA is a bicyclic nucleic acid, where a ribonucleoside is linked between the 2′-oxygen and the 4′-carbon atoms with a methylene unit.

LNA is a bicyclic RNA analogue, in which the ribose moiety in the sugar-phosphate backbone is structurally constrained by a methylene bridge between the 2′-oxygen and the 4′-carbon atoms (Obika et al. 1998, Koskhin et al. 1998, Singh et al. 1998).

“The first analogues of LNA (Locked Nucleic Acids): Phosphorothioate-LNA and 2′-thio-LNA” by Jesper Wengel et al; Bioorganic & Medicinal Chemistry Letters; Volume 8, Issue 16, 18 Aug. 1998, Pages 2219-2222 discloses LNA (Locked Nucleic Acids, 1, X═O, Y═O) as a novel oligonucleotide analogue capable of recognizing complementary DNA and RNA with unprecedented thermal affinities. Synthesis of the first chemically modified LNA analogues was reported.

“Locked Nucleic Acid: A Potent Nucleic Acid Analog in Therapeutics and Biotechnology” by Jan Stenvang Jepsen et al., Oligonucleotides, April 2004, 14(2): 130-146. doi:10.1089/1545457041526317 discloses that locked nucleic acid (LNA) is a class of nucleic acid analogs possessing very high affinity and excellent specificity toward complementary DNA and RNA; and LNA oligonucleotides have been applied as antisense molecules both in vitro and in vivo.

The pre-organized conformation of the LNA nucleoside was predicted to be N-type sugar puckering, characteristic for A-type double helices, such as RNA-RNA duplexes. This assumption has been confirmed by. NMR solution studies and X-ray crystallographic analysis. The preliminary LNA® nucleoside spectra demonstrated the fixed N-type conformation of LNA® (Koskhin et al. 1998, Singh et al. 1998).

Subsequent NMR studies have analyzed that the fixed N-type (3′-endo) conformation of the LNA nucleoside, together with enhanced stacking of the nucleobases results in higher thermal stability of LNA-containing duplexes.

“Synthesis and restricted furanose conformations of three novel bicyclic thymine nucleosides: a xylo-LNA nucleoside, a 3′-O,5′-C methylene-linked nucleoside, and a 2′-O, 5′-C-methylene-linked nucleoside” by Vivek K. Rajwanshi, Jesper Wengel et al.; Center for Synthetic Bioorganic Chemistry, Department of Chemistry, University of Copenhagen, Universitetsparken 5, Copenhagen, Denmark; Accepted 16 Apr. 1999; discloses synthesis of three novel classes of conformationally restricted nucleoside analogues, all containing a bicyclic pentofuranose moiety and hydroxy groups positioned at the 3′ and 5′ positions, allowing the formation of 3′O- to 5′O-linked oligonucleotide analogues and 5′-phosphorylated nucleoside derivatives thus mimicking the natural regiochemistry. Solution-phase conformational analysis showed the bicyclic nucleosides (as depicted therein as 8, 9, 14, 15, 17 and 19) to exist predominantly in N-type furanose conformation.

U.S. Pat. No. 7,034,133, U.S. Pat. No. 7,053,207, U.S. Pat. No. 6,268,490 and Michael Petersen et al in J. Am. Chem. Soc., 2002, 124 (21), pp 5974-5982 disclose locked nucleic acids (LNAs) containing one or more 2′-O, 4′-C locked nucleosides and related compounds and their methods of synthesis.

“Preparation and properties of 2′,5′-linked oligonucleotide analogues containing 3′-O, 4′-C-methyleneribonucleosides” by. Satoshi Obika and Takeshi Imanishi et al.; Bioorganic & Medicinal Chemistry Letters; Volume 9, Issue 4, 22 Feb. 1999, Pages 515-518; discloses that bicyclic nucleoside analogues 3′-O,4′-C-methyleneuridine and -5-methyluridine, were successfully incorporated into oligonucleotides via connection with 2′,5′-phosphodiester linkage; and further, hybridization behavior and nuclease stability of the modified oligonucleotides were investigated. The phosphoramidite building blocks were prepared from the corresponding 5′-O-dimethyltrityl derivatives. The modified units were successfully incorporated in 3′-5′ phosphodiester linked oligonucleotides using the standard phosphoramidite protocol on the DNA synthesizer.

“Facile synthesis and conformation of 3′-O,4′-C-methyleneribonucleosides” by Satoshi Obika, Ken-ichiro Moho et al.; Chem. Commun., 1999, 2423-2424; Satoshi Obika et al; “Synthesis and conformation of 3′,4′-BNA monomers, 3′-O,4′-C-methyleneribo nucleosides” in Tetrahedron, Volume 58, Issue 15, 8 Apr. 2002, Pages 3039-3049; and “Synthesis and conformation of 3′-O,4′-C-methyleneribonucleosides, novel bicyclic nucleoside analogues for 2′,5′-linked oligonucleotide modification” by Satoshi Obika et al. disclose novel bicyclic nucleoside analogues for 2′,5′-linked oligonucleotide modification and their synthesis.

U.S. Pat. No. 7,153,954 relates to large scale preparation of LNA phosphoramidites comprising phosphitylation of the 3′-OH group of an LNA monomer with a 2-cyanoethyl-N,N,N′,N′-tetra-substituted phosphoramidite in the presence of a nucleophilic activator, e.g. 2-cyanoethyl-N,N,N′,N′-tetraisopropylphosphoramidite and 4,5-dicyanoimidazole. The method is faster and more cost efficient than previously known methods.

WO0220537 relates to linker phosphorarhidites for oligonucleotide synthesis and preparation thereof.

WO 2005/023825 discloses bicyclic nucleosides and oligomeric compounds comprising at least one such nucleoside. A bicyclic sugar moiety that has been prepared and studied has the bridge going from the 3′-hydroxyl group via a single methylene group to the 4′-carbon of the sugar ring thereby forming a 3′-C, 4′-O-oxymethylene linkage. In some embodiments, each of the high modified nucleoside is, independently, a bicyclic sugar modified nucleoside, a 2′-F modified nucleoside.

“Current protocols in Nucleic acid Chemistry”; Supplement 35; 1.0.1-1.0.3, December 2008; Published online December 2008 in Wiley Interscience (www.interscience.wiley.com);Chapter 1; Synthesis of modified Nucleosides; discloses the modified nucleosides containing reactive functionality, of which are 2-fluoro-2-deoxyinosine derivative, 2′-fluoro-2′,3′-dideoxyadenosine and 2′-deoxy-2′-fluoroarabinonucleosides. Oligonucleotides containing these modified bases form stable heteroduplexes with RNA, which is important to antisense applications.

In view of the above trend, the present inventors propose to study the structural preferences of 2′-5′ linked ribo/deoxyribonucleic acid duplexes with RNA by introduction of appropriate structural regulators/locks. Study of structurally isomeric, conformationally constrained 2′-5′ linked ribo/deoxyribonucleic acids have not been reported for S-type (2′-endo or 3′-exo) locked uridine; N-type (2′-exo or 3′-endo) locked uridine; and 2′-O-phosphoramidite of 3′-fluoro-3′-deoxy uridine monomer unit, but the synthesis of monomeric nucleoside units locked either in 3′-endo and 2′-endo geometries are known.

The present inventors thus synthesize conformationally locked novel N-type (3′-endo) 2′-5′ linked ribo/deoxyribonucleic acids; 3′-fluoro-2′-5′ linked nucleic acid; 6′-(O-Dimethoxytrityl)-hydroxymethyl-4′-hydroxy-3′(purinyl/pyrimidinyl)-cyclohexene-2′-O-phosphoramidite and also propose the synthesis of S-type (3′-endo) (wherein n=1,2,3) 2′-5′ linked ribo/deoxyribonucleic acids by novel methods described herein below.

### OBJECTS OF THE INVENTION

The main object of the invention is to provide novel 2′-O-phosphoramidite of N-type (3′-endo) locked nucleosides and 2′-O-phosphoramidite of S-type (2′-endo) locked nucleosides and-process of preparation thereof.

Another object of the invention is to provide unlocked nucleosides such as 2′-O-phosphoramidite of 3′-fluoro-3′-deoxy-xylo/ribo uridine and 6′-(O-Dimethoxytrityl)-hydroxymethyl-4′-hydroxy-3′(purinyl/pyrimidinyl)-cyclohexene-2′-O-phosphoramidite and process for synthesis thereof.

Further object of the invention is to incorporate the synthesized monomers into 2′-5′-linked oligomers to delineate the structural requirements of duplexes of these oligomers with 3′-5′ RNA and their biophysical implications on the stability of the said duplexes towards development into therapeutic oligomers.

Yet another object of the invention is the use of the synthesized molecular entities for antisense, siRNA (small interfering RNA) based drug development.

### SUMMARY OF THE INVENTION

The present invention discloses 2′-O-phosphoramidite locked nucleoside and unlocked nucleoside, their synthesis and oligomer comprising nucleosides incorporated into 2′-5′-linked oligomers to delineate the structural requirements of 2′-5′ RNA/DNA: 3′-5′ RNA duplexes and for use in antisense applications.

Accordingly, the present invention discloses 2′-O-phosphoramidite of N-type and S-type locked nucleosides of formula I

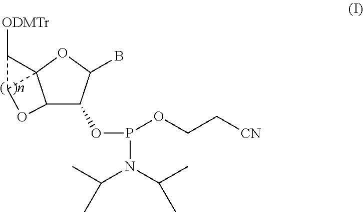

wherein the dotted line represents 3′-O,5′-C or 3′-O,4′-C-oxymethylene linkage respectively; and wherein n=1, 2 or 3 and B is selected from a pyrimidine or a purine nucleic acid base.

Thus the present invention also discloses process of synthesis of novel 2′-O-phosphoramidite of N-type (3′-endo) and 2′-O-phosphoramidite of S-type (2′-endo) locked nucleoside and 2′-5′-linked ribo/deoxyribonucleic acid oligomers comprising 2′-O-phosphoramidite of N-type and S-type locked nucleosides of formula I. The nucloesides are compatible for automated synthesis of N-type (3′-endo) and S-type locked (wherein n=1,2,3) 2′-5′-linked ribo/deoxyribonucleic acid (2′-5′-linked oligomer).

In further aspect of the invention is disclosed phosphoramidite unlocked nucleosides of Formula VI,

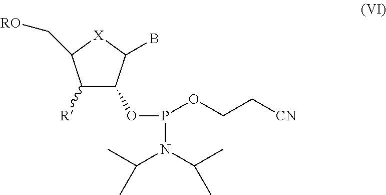

Thus present invention also discloses the process of synthesis of phosphoramidite unlocked nucleosides and oligomers comprising the nucleosides of Formula VI.

In another embodiment of the invention is disclosed unlocked nucleosides such as 2′-O-phosphoramidite of 3′-fluoro-3′-deoxy-xylo/ribo-uridine and 6′-(O-Dimethoxytrityl)-hydroxymethyl-4′-hydroxy-3′ (purinyl/pyrimidinyl)-cyclohexene-2-O-phosphoramidite; process for preparing the same and further their incorporation into 2′-5′-linked oligomers to delineate the structural requirements of duplexes of these oligomers with 3′-5′ RNA.

In another embodiment 2′-O-phosphoramidite of N-type locked uridine is prepared starting from D-glucose.

In one embodiment is disclosed, a synthetic method for the preparation of S-type (2′-endo structures) locked uracil monomer unit, wherein n=1,2,3 and B═U starting from uridine, followed by the preparation of 2′-O-phosphoramidite of S-type locked uridine.

In another embodiment, the invention provides 3′-fluoro-3′-deoxy-xylofuranosyl-2′-O-phosphoramidite prepared by a process starting from D-glucose and 3′-fluoro-3′-deoxy-ribofuranosyl -2′-O-phosphoramidite from 3′-deoxy-3′-fluoro-5-hydroxy-ribofuranosyl uridine, amicable for the synthesis of respective 2′-5′-linked oligomers using automated DNA synthesis machine.

In yet another embodiment of the invention, 6′-(O-Dimethoxytrityl)-hydroxymethyl-4′-hydroxy-3′(purinyl/pyrimidinyl)-cyclohexene-2′-O-phosphoramidite is prepared starting from racemic Diels-Alder adduct.

### DETAILED DESCRIPTION OF THE INVENTION

The invention will now be described in detail in connection with certain preferred and optional embodiments, so that various aspects thereof may be more fully understood and appreciated.

As used herein, the term oligomer or oligonucleotide refers to two or more contiguous molecular entities, synthesized according to the present invention in 2′-5′-phosphodiester linkage. The 2′-5′-linked oligomers derived from one or more of the nucleoside analogues in combination with the naturally-occurring nucleosides are also within the scope of the present invention.

Accordingly, the present invention provides 2′-O-phosphoramidite of N-type and S-type locked nucleosides of formula I,

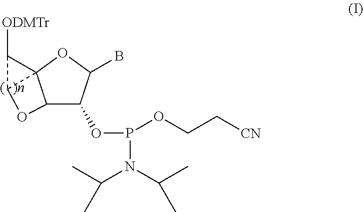

wherein the dotted line represents 3′-O,5′-C or 3′-O,4′-C-oxymethylene linkage respectively; and wherein n=1, 2 or 3 and B is selected from a pyrimidine or a purine nucleic acid base.

The present invention provides 2′-O-phosphoramidite of N-type (3′-endo) and 2′-O-phosphoramidite of S-type (2′-endo) locked uridine monomer units as well as 2′-5′-linked N-type and 2′-5′-linked S-type (where n=1,2,3) oligomers derived from these phosphoramidite monomer unit or comprising one or more of the aforementioned compounds.

Further, the invention provides the unlocked nucleosides such as 2′-O-phosphoramidite of 3′-fluoro-3′-deoxy xylo/ribo-uridine and 6′-(O-Dimethoxytrityl)-hydroxymethyl-4′-hydroxy-3′(purinyl/pyrimidinyl)-cyclohexene-2′-O-phosphoramidite; their synthesis and their incorporation into 2′-5′-linked oligomers.

Furthermore, the present invention involves the use of the synthesized 2′-5′-linked oligomers in the formation of duplexes with 3′-5′ RNA.

Accordingly, the present invention provides 2′-O-phosphoramidite of N-type locked nucleoside of formula (II),

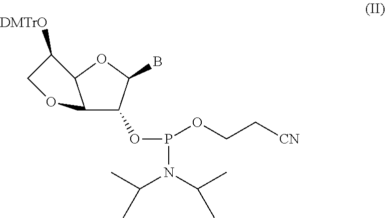

Wherein B is pyrimidine or purine nucleic acid base, preferably a pyrimidine base, uracil.

Further, the present invention provides novel N-type (3′-endo) 2′-5′ linked ribo/deoxyribonucleic acid oligomer, represented by formula (III), comprising 2′-5′O-phosphoramidite of N type (3′endo) nucleosides of Formula II

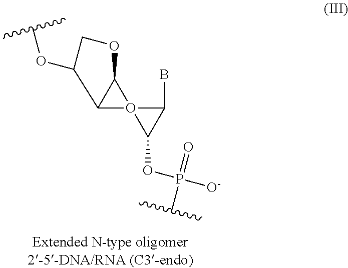

wherein B is pyrimidine or purine nucleic acid base, preferably a pyrimidine base, uracil.

Accordingly, 2′-5′-linked ribo/deoxyribonucleic acid oligomers comprising nuclosides of Formula II comprises a sequence selected from the group comprising of following sequences, wherein U^{N} is N-type locked nucleoside monomer

5′-CAC CAT TGT CAC AC [symbol] CCA-2′
5′-CAC CAT TG [symbol] CAC AC [symbol] CCA-2′
5′-CAC CA [symbol] TG [symbol] CAC AC [symbol] CCA-2′
5′-CCT CTT ACC TCA GT [symbol] ACA-2′
5′-CCT CTT ACC [symbol] CA GT [symbol] ACA-2′
5′-CCT CT [symbol] ACC [symbol] CA GT [symbol] ACA-2′

Accordingly, the process for preparing 2′-O-phosphoramidite of N-type (2′-exo or 3′-endo) locked nucleoside of formula (II), comprisies protecting glucose molecule with isopropylidine group and converting 1,2 and 5,6-isopropylidine protected glucose to C3-epimer through oxidation and reduction steps; followed by tosylation at C3-position and removal of 5,6 isopropylidine protection to give free diol, which on treatment with base to give intermediate N-type locked ribose sugar. Further, introduction of nucleobase (preferably uracil) and deprotection of 2′-hydroxy group, followed by the reaction of deprotected intermediate with chloro (2-cyanoethoxy)-N,N-diisopropyl amino)-phosphine to give 2′-O-phosphoramidite of N-type locked uridine.

The synthetic route for the preparation of 2′-O-phosphoramidite of N-type locked nucleoside (preferably N-type locked uridine, wherein B═U) and further to N-type (2′-exo or 3′-endo) locked 2′-5′ linked ribo/deoxyribonucleic acid oligomer is as depicted in scheme 1.

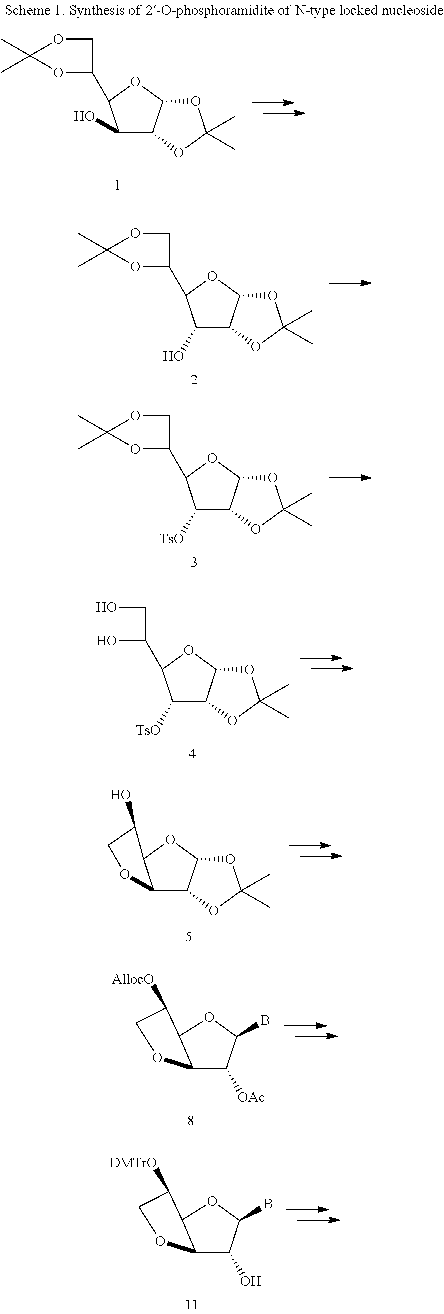

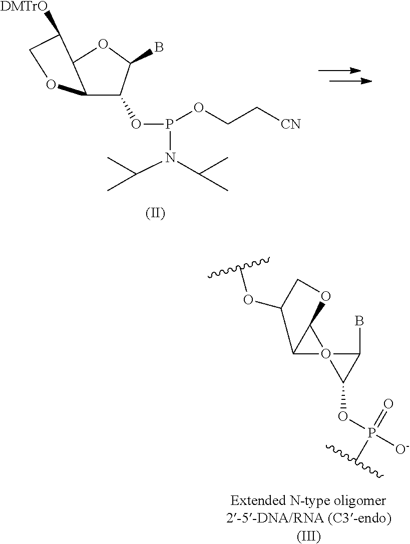

According to the preferred embodiment and in accordance with the above scheme, the invention provides a detailed synthetic route starting from 3,6-anhydro-5-hydroxy-1,2-isopropylidene-Glucofuranose (5) to obtain 2′-O-phosphoramidite of N-type (2′-exo or 3′-endo) locked purinyl/pyrimidinyl ribonucleoside (preferably N-type locked uridine, wherein B═U) which comprises the following steps:
reacting of 3,6-anhydro-5-hydroxy-1,2-isopropylidene-Glucofuranose (5) with allyloxycarbonyl chloride to obtain 5-O-allyloxycarbonyl-3,6-anhydro-1,2-isopropylidine-Glucofuranose (6):

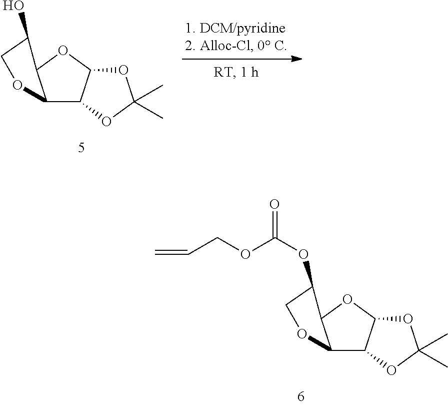

(ii) treating compound of formula (6) as obtained in step (i) with a mixture of acetic acid, acetic anhydride and sulfuric acid to give 1,2-di-O-acetyl-3,6-anhydro-5-O-allyloxycarbonyl-α,β-Glucofuranose (7);

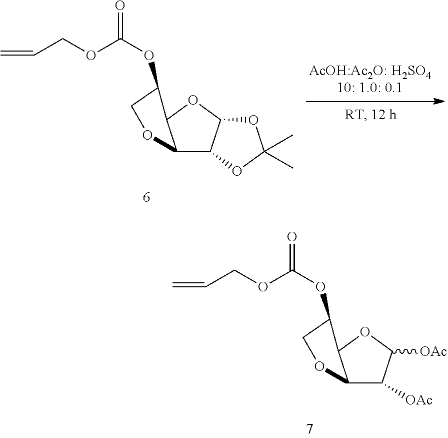

(iii) reacting of compound of formula (7) as obtained in step (ii) with nucleobase in presence of N,O-Bis(trimethylsilyl)acetamide (BSA) and Trimethylsilyl Trifluoromethanesulfonate (TMSOTf) to obtain 2′-O-acetyl-3′,6′-anhydro-5′-O-allyloxycarbonyl-nucleoside (8);

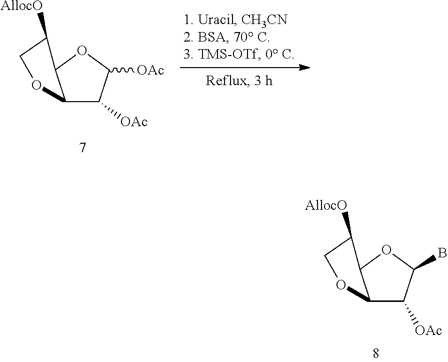

(iv) deprotecting of allyloxycarbonyl group at 5′-position of compound (8) as obtained in step (iii) in presence of Tris(dibenzylidene acetone) dipalladium [Pd₂(dba)₃] catalyst to obtain 5′-hydroxy-2′-O-acetyl-3′,6′-anhydro-nucleoside (9);

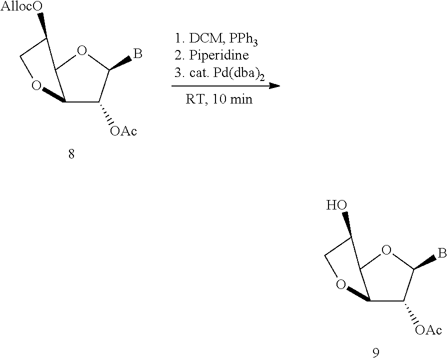

(v) treating 5′-hydroxy-2′-O-acetyl-3′,6′-anhydro-nucleoside (9) as obtained in step (iv) with 4,4′-dimethoxytritylchloride in presence of pyridine (dry) at room temperature for 20-25 hrs to give 5′-O-dimethoxytrityl-2′-O-acetyl-3′,6′-anhydro-nucleoside (10);

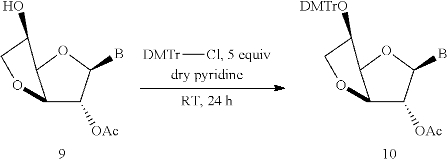

(vi) hydrolying of compound (10) as obtained in step (v) with aqueous ammonia in presence of methanol to yield 5′-O-dimethoxytrityl-2′-hydroxy-3′,6′-anhydro-nucleoside (11); and

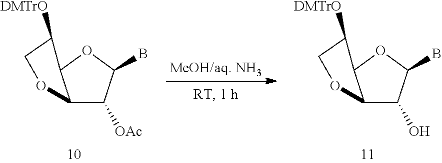

(vii) treating compound (11) as obtained in step (vi) with chloro(2-cyanoethoxy)-N,N-diisopropyl amino)-phosphine in presence of Diisopropylethylamine (DIPEA) to give final compound 5′-O-Dimethoxytrityl-3′-O,5 ′-C-methylene uridine) xylonucleoside-2′-O-phosphoramidite, which is 2′-O-phosphoramidite of N-type (2′-exo or 3′-endo) locked nucleoside (II).

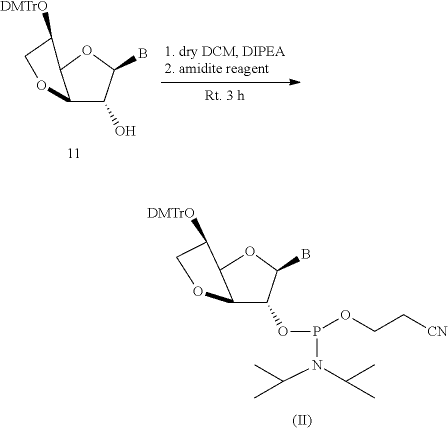

Further, 2′-5′ linked oligomer (N-type (2′-exo or 3′-endo) locked 2′-5′ linked ribo/deoxyribonucleic acid) (III) is prepared from the synthesized phosphoramidite monomer unit (8) using automated DNA synthesis machine (DNA synthesizer).

The 3,6-anhydro-5′-hydroxy-1,2-isopropylidene-Glucofuranose (5) used in scheme 1 is prepared by a process comprising the steps of:
(a) protecting 1,2 and 5,6-position of glucose by isopropylidine group to give 1,2 and 5,6-isopropylidine protected glucose (1)

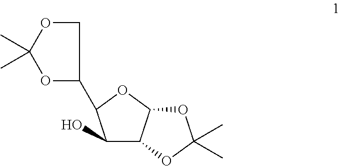

(b) converting 1,2 and 5,6-isopropylidine protected glucose(1) as obtained in step (a) to C3-epimer (2) through oxidation and reduction steps;

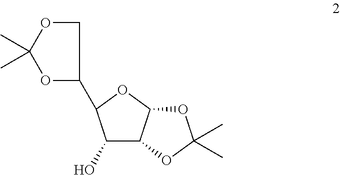

(c) tosylating at C3-position of compound (2) to give compound (3);

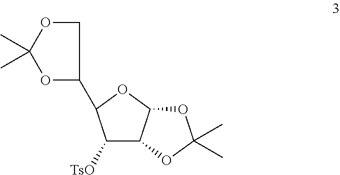

(d) removing 5,6-isopropylidine protection of compound (3) as obtained in step (c) to give free diol (4);

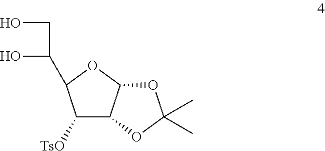

(e) treating compound (4) as obtained in step (d) with base to give intermediate N-type locked ribose sugar (5);

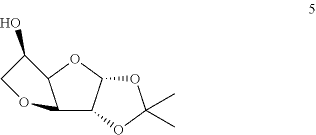

In another embodiment, the present invention provides 2′-O-phosphoramidite of S-type locked nucleoside of formula IV,

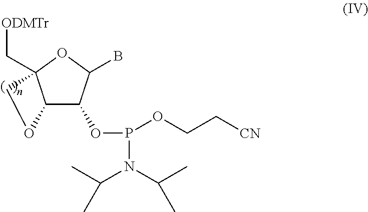

wherein n=1,2,3 and B is pyrimidine or purine nucleic acid base, preferably a pyrimidine base, uracil.

Further, the present invention provides S-type (2′-endo) 2′-5′ linked ribo/deoxyribonucleic acid oligomer, represented by formula (V), comprising 2′-O-phosphoramidite S type (2′-endo) locked nucleosides of Formula IV.

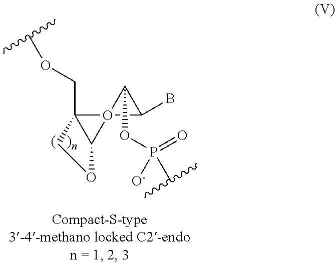

wherein n, and B have the same meaning as given above.

Accordingly, 2′-5′-linked ribo/deoxyribonucleic acid oligomersS-type (2′-endo) locked 2′-5′ linked ribo/deoxyribonucleic acid oligomer of Formula V comprises a sequence selected from the group comprising of following sequences, wherein U^{S} is S-type locked nucleoside monomer

5′-CAC CAT TGT CAC AC [symbol] CCA-2′
5′-CAC CAT TG [symbol] CAC AC [symbol] CCA-2′
5′-CAC CA [symbol] TG [symbol] CAC AC [symbol] CCA-2′
5′-CCT CTT ACC TCA GT [symbol] ACA-2′
5′-CCT CTT ACC [symbol] CA GT [symbol] ACA-2′
5′-CCT CT [symbol] ACC [symbol] CA GT [symbol] ACA-2′

In a further embodiment of the invention, there is provided a synthetic method for the preparation of 2′-O-phosphoramidite of S-type locked nucleoside of formula (IV), more specifically 2′-O-phosphoramidite of S-type locked uridine, if B═U starting from uridine, compatible for automated synthesis of 2′-5′-linked oligomers (S-type 2′-5′-linked ribo/deoxyribonucleic acid) of formula V.

Accordingly, the process for preparing 2′-O-phosphoramidite of S-type (2′-endo structures) locked uridine monomer unit where n=1 and B═U comprises the following steps: conversion of 2′,3′-protected uridine to 5′-aldehyde and then subjected to hydroformylation at C-4′; reduction; tosylation reaction, followed by introduction of 4,4′-dimethoxytrityl (DMTr) group at 5′-position by reacting with 4,4′-dimethoxytrityl chloride to give the corresponding dimethoxytrityl compound and ring closure to yield S-type locked uridine monomer unit, followed by the preparation of 2′-O-phosphoramidite of S-type locked uridine using standard procedure.

Further, S-type (2′-exo or 3′-endo) locked 2′-5′-linked ribo/deoxyribonucleic acid is prepared from the synthesized 2′-O-phosphoramidite of S-type locked uridine using automated DNA synthesis machine.

The synthetic route for the preparation of 2′-O-phosphoramidite of S-type locked uridine, where n=1 and B═U, is as depicted in scheme 2.

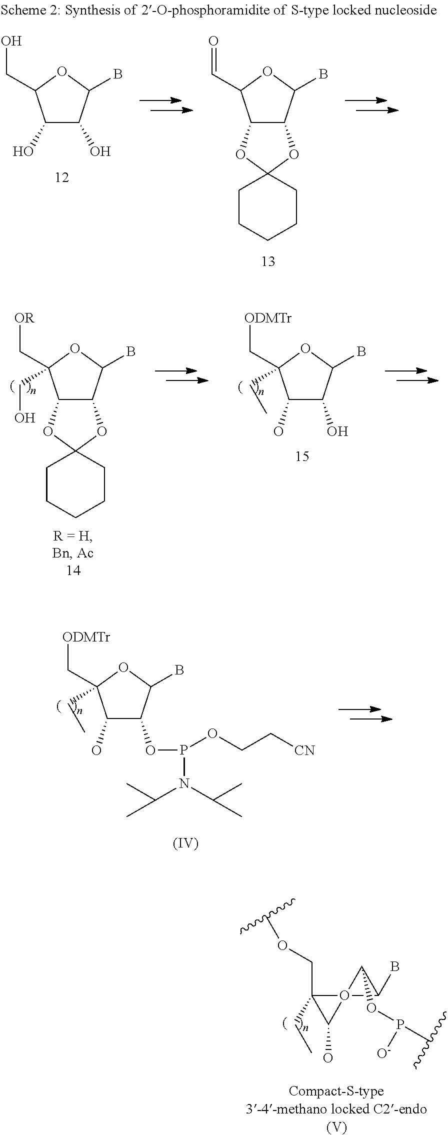

5′-O-Dimethoxytrityl-2′-hydroxy-3′-O,4′-C-methylene (uridine) ribonucleoside (15), obtained is converted to 5′-O-Dimethoxytrityl-3′-O,4′-C-methylene (uridine) ribonucleoside-2′-O-phosphoramidite of formula (IV), by a reported procedure in literature.

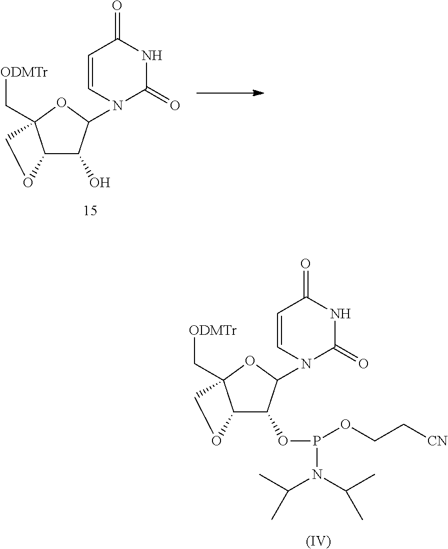

The preparation of 2′-O-phosphoramidite of S-type locked uridine as in scheme 2 comprising following steps:
(A) protecting 2′,3′ position of uridine by cyclohexylidene group to give 2′-3′-O-cyclohexylidene-uridine, followed by oxidizing 5′-OH group of 2′,3′-protected nucleoside to give 5′-aldehydo-2′,3′-O-cyclohexylidene-nucleoside (13);

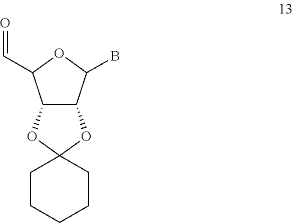

(B) hydroformylating C-4′position and reducing 5′-aldehyde group of compound (13) as obtained in step (A) to give 4′-hydroxymethyl-2′,3′-O-cyclohexylidene-nucleoside (14);

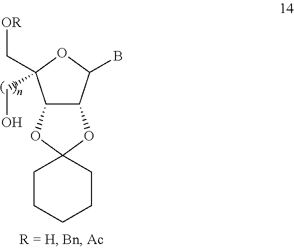

(C) tosylating 4′-position, deprotecting 2,3 position to obtain 4′-p-toluenesulphonyl-methyl-uridine and reacting 4′-p-toluenesulphonyl-methyl-uridine with optimized of 4,4′-dimethoxytritylchloride, followed by ring closure to yield 5′-O-DMTr-O3′-4′-methylene bridged nucleoside (15); and

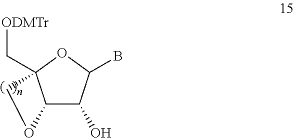

(D) converting compound (15) as obtained in step (C) to 2′-O-phosphoramidite of S-type locked nucleoside (IV), wherein, n=1 and B═U

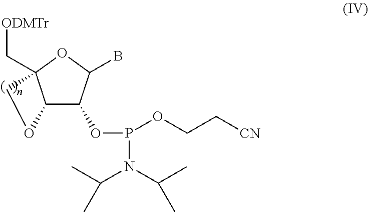

Further, S-type (2′-exo or 3′-endo) locked 2′-5′-linked ribo/deoxyribonucleic acid is prepared from the synthesized 2′-Ophosphoramidite of S-type locked uridine using automated DNA synthesis machine.

In an alternative embodiment, the process for preparing 2′-O-phosphoramidite of S-type locked nucleoside comprising preparing O3′-4′-methylene-bridged uridine from 4′-p-Toluenesulphonylmethyl-uridine, followed by reacting with DMTr to yield 5′-O-DMTr-O3′-4′-methylene bridged uridine (15) and finally converting compound (15) to 2′-O-phosphoramidite of S-type locked nucleoside (IV), wherein, n=1 and B═U.

In yet another aspect, the present invention provides 2′-O-phosphoramidite unlocked nucleoside of formula VI and process of preparations thereof.

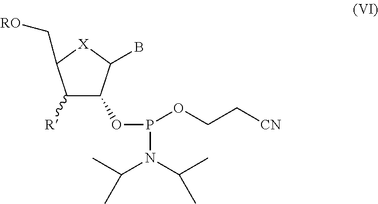

wherein X═O or (C═C); R′═F or H; R=Dimethyltrityl group (DMTr) and B=pyrimidine or purine nucleic acid base, preferably pyrimidine base, uracil.

Further, the invention provides 2′-5′-linked nucleic acid oligomer of formula VII comprising nuclosides of Formula VI,

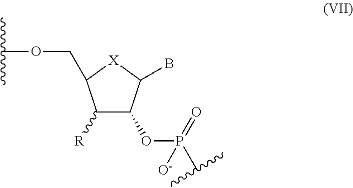

prepared by using the synthesized phosphoramidite monomer unit (VI), wherein X═O or (C═C); R′═F or H; and B=pyrimidine or purine nucleic acid base, preferably pyrimidine base, uracil.

Accordingly, in the present invention provides 3′-fluoro-3′-deoxy-2′-O-phosphoramidite monomer of formula VI, wherein R′═F and X═O represented by compound of formula VIII as herein below,

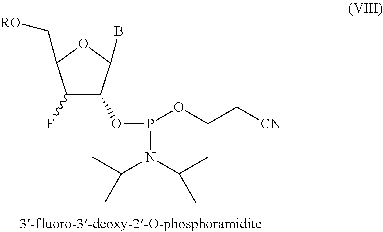

wherein R and B have the same meaning as given above.

According to the invention, 3′-fluoro-3′-deoxy-2′-O-phosphoramidite monomer of formula VIII is selected from 3′-fluoro-3′-deoxy-ribofuranosyl-2′-O-phosphoramidite of formula (VIIIa) and 3′-fluoro-3′-deoxy-xylofuranosyl-2′-O-phosphoramidite of formula (VIIIb) as represented herein below.

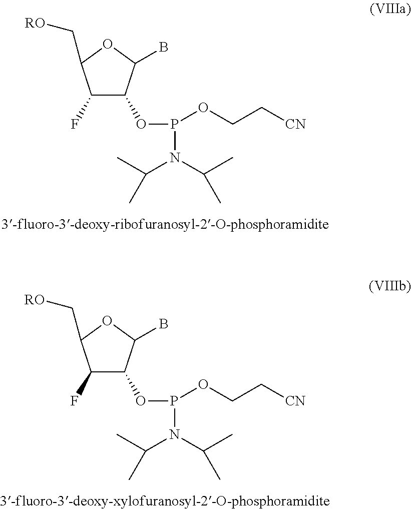

wherein R and B have the same meaning as herein above.

Further, in one embodiment the present invention provides novel 3′-fluoro-2′-5′-linked nucleic acid oligomer of formula VII, wherein R′═F and X═O, represented by compound of formula IX,

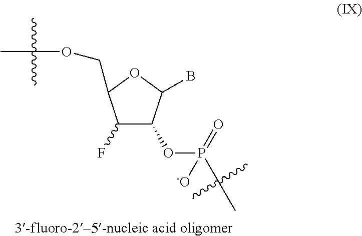

wherein B is a pyrimidine or purine nucleic acid base, preferably a pyrimidine base, uracil.

Compounds VIII described herein above are amicable for the synthesis of 3′-fluoro-2′-5′-linked nucleic acid oligomer of formula IX.

According to the invention, compound of formula (IX) is selected from 3′-fluoro-2′-5′-linked ribo/deoxy-ribonucleic acid (2′-5′-RNA/DNA) of formula (IXa) and xylo/deoxy-xylonucleic acid (2′-5′-XNA/dXNA) of formula (IXb), which are synthesized from compound of formula (VIIIa) and (VIIIb), respectively,

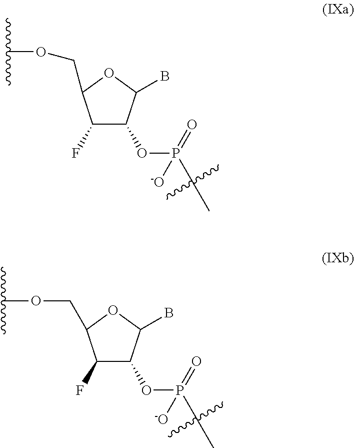

wherein B has the same meaning as given above.

Accordingly 3′-fluoro-2′-5′-linked ribo/deoxy-ribonucleic acid (2′-5′-RNA/DNA) oligomer of Formula (IXa) and (Ixb) comprises a sequence selected from the group comprising of the following sequences, wherein U^{rF} is fluoro ribonucleoside monomer

5′-CAC CAT TGT CAC AC [symbol] CCA-2′
5′-CAC CAT TG [symbol] CAC AC [symbol] CCA-2′

Accordingly 3′-fluoro-2′-5′-linked xylo/deoxy-xylonucleic acid (2′-5′-XNA/dXNA) oligomer comprises a sequence of Formula (IXa) and (Ixb) selected from the group comprising of following sequences, wherein U^{xF} is fluoro xylonucleoside monomer

5′-CAC CAT TGT CAC AC [symbol] CCA-2′
5′-CAC CAT TG [symbol] CAC AC [symbol] CCA-2′

Further, the invention provides a process for the synthesis of 2′-O-phosphoramidite of 3′-fluoro-3′-deoxy-xylo-uridine (VIIIb) by simple reaction sequences starting from D-glucose.

According to the invention, 1,2 and 5,6-isopropylidine protected glucose, which is prepared from D-glucose by one step, was converted to C3-epimer through oxidation and reduction steps. Tosylation reaction at C3-position was performed using tosyl chloride, followed by displacement of tosyl group with fluoride anion to give the required intermediate for converting it to 3′-fluoro-3′-deoxy-xylofuranosyl-2′-O-phosphoramidite by using standard procedure.

The synthetic route for the preparation of 3′-fluoro-3′-deoxy-xylofuranosyl-2′-O-phosphoramidite (VIIIb) is as depicted below in Scheme 3.

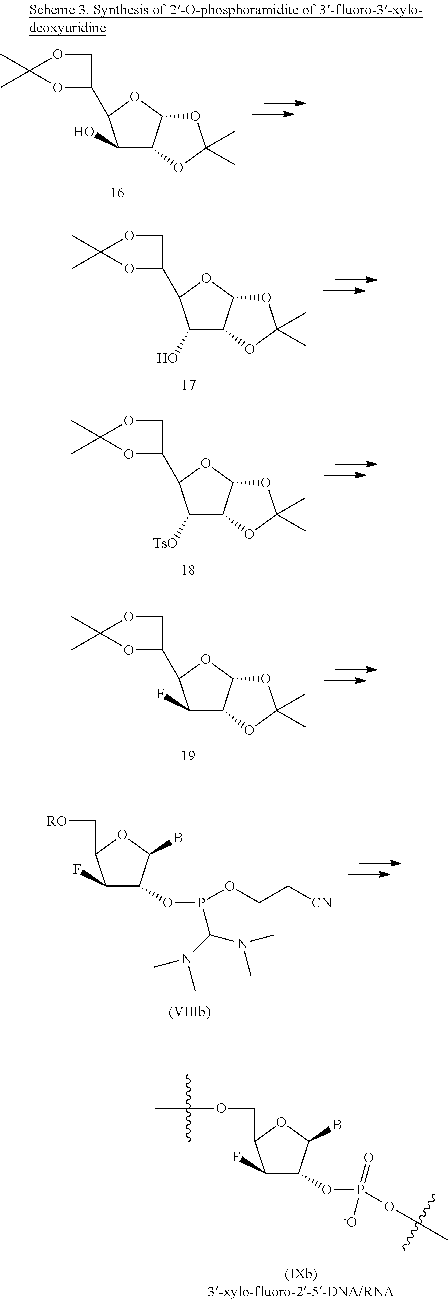

Further, 3′-fluoro-3′-deoxy-xylofuranosyl-2′-O-phosphoramidite or 5′-O-(4,4′-dimethoxytrityl)-β-D-xylofuranosyl 3′-deoxy-3′-fluoro-uridinyl -2-O-phosphoramidite of formula (VIIIb) is incorporated into 2′-5′-linked oligomers using automated DNA synthesis machine to prepare 3′-fluoro-2′-5′-linked xylo/deoxyxylonucleic acid (IXb).

The sequence of the above process steps is as follows:
(I) protecting 1,2 and 5,6-position of glucose by isopropylidine groups and converting isopropylidine protected glucose (16) to C3-epimer (17) through oxidation and reduction steps;
(II) tosylating at C3-position to obtain (18);
(III) displacing tosyl group of compound 18 as obtained in step (II) with fluoride anion to give C3-fluoro intermediate (19);
(IV) converting fluoro intermediate 19 as obtained in step (III) to 3′-fluoro-3′-xylofuranosyl-3′-deoxy-2′-phosphoramidite (VIIIb);and synthesizing 3′-fluoro-2′-5′-linked xylo/deoxy-xylonucleic acid (IXb) using 3′-fluoro-3′-deoxy-xylofuranosyl-2′-O-phosphoramidite (VIIIb).

Furthermore, a process for preparation of 3′-fluoro-3′-deoxy-ribofuranosyl-2′-O-phosphoramidite (VIIIa), wherein B═U and R=DMTr, comprises the following steps:
(V) reacting 3′-deoxy-3′-fluoro-5′-hydroxy ribofuranosyl uridine (20) with 4,4′-Dimethoxy tritylchloride, in the presence of catalytic amount of 4-dimethylaminopyridine dissolved in pyridine to obtain 5′-O-dimethoxytrityl-3′-deoxy-3′-fluoro-ribofuranosyl uridine (21);

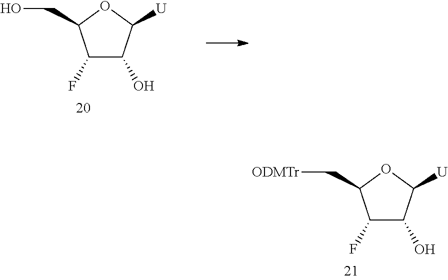

(VI) dissolving compound (21) as obtained in step (V) in dry DCM followed by the addition of diisopropyl ethyl amine and chloro (2-cyanoethoxy) (N,N-O-diisopropylamino)-phosphine to obtain 3′-fluoro-3′-deoxy-ribofuranosyl-2′-O-phosphoramidite or 5 ′-O-(4,4′-dimethoxytrityl)-β-D-ribofuranosyl 3′-deoxy-3′-fluoro-uridinyl-2-O-phosphoramidite of formula (VIIIa).

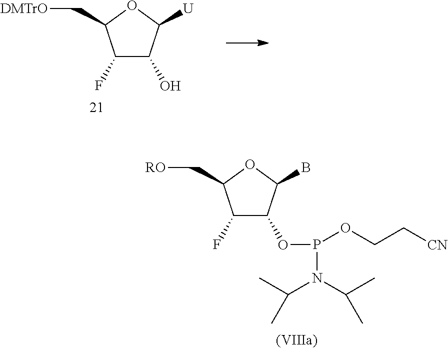

Further, the synthesized 3′-fluoro-3′-deoxy-ribofuranosyl-2′-O-phosphoramidite or (VIIIa) is converted into 3′-fluoro-2′-5′-linked ribo/deoxy-ribonucleic acid (IXa).

In a further embodiment the present invention provides 6′-(O-Dimethoxytrityl)-hydroxymethyl-4′-hydroxy-3′ (purinyl/pyrimidinyl)-cyclohexene-2′-O-phosphoramidite of formula VI, wherein R′′H and X═C═C, represented by compound of formula X as herein below,

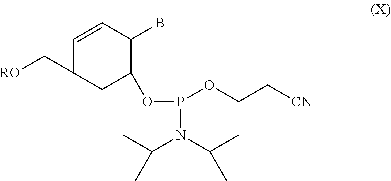

wherein R=DMTr.

Further, disclosed is a process for the synthesis of compound of formula (X) by simple reaction sequences starting from racemic Diels-Alder adduct.

In another embodiment 2′-5′-linked ribo/deoxyribonucleic acid oligomers of formula VII, wherein R′═H and X═(C═C), is provided by the present invention.

According to the invention, in the process of preparing 6′-(O-Dimethoxytrityl)-hydroxymethyl-4′-hydroxy-3′(purinyl/pyrimidinyl)-cyclohexene-2′-O-phosphoramidite, the racemic Diels-Alder adduct of formula 22 in phosphate buffer (pH=7.2) was treated with lipase (Candida cylindracea) and stirring at 60° C. for 72 hr resulted in almost 45% hydrolysis of the acetate (Scheme 4). This hydrolysis was found to be highly enantioselective (>95% based on NMR), yielding only a single enantiomer of formula 23 and leaving the acetate of formula 22 enriched in the other enantiomer (>95% based on NMR).Treatment of the alcohol of formula 23 with a mixture of ammonia and sodium in THF/EtOH yielded product 24. Treatment with benzoyl chloride, followed by ketal hydrolysis then gave lactone 26. mCPBA oxidation yielded an inseparable mixture of 27 and 28, which on reduction yielded an inseparable mixture of 29 and 30 along with product 31. Treatment of the mixture of 29 and 30 with benzaldehydedimethylacetal yielded the benzylidene derivative 32 from 29, leaving triol 30 unreacted. Triol 30 was confirmed by converting it to its triacetate derivative 33 for characterization. Stereochemically pure 30 can be converted to the corresponding DMTr-protected base-containing phosphoramidite X by standard procedures, which can be used in the synthesis of 2′-5′-linked DNA/RNA.

The synthetic route for the preparation of 6′-(O-Dimethoxytrityl)-hydroxymethyl-4′-hydroxy-3′(purinyl/pyrimidinyl)-cyclohexene-2′-O-phosphoramidite of formula X is as depicted in scheme 4.

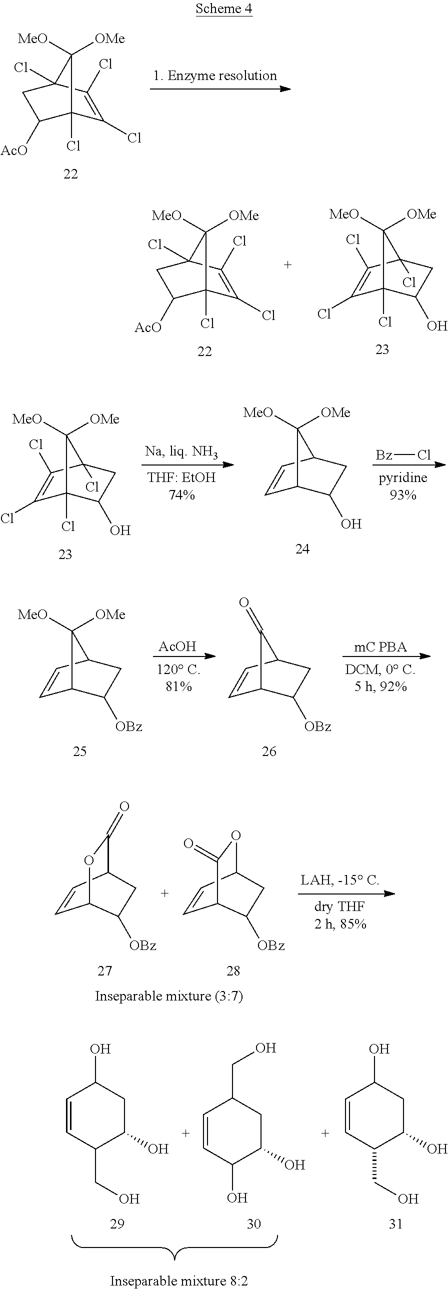

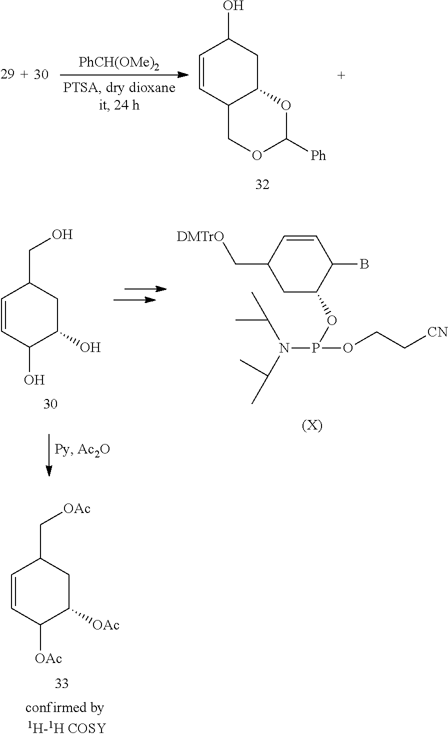

The sequence of the above process steps is as follows:
(1) hydrolyzing racemic Diels-Alder adduct of formula 22 enantioselectively (45%) in phosphate buffer (pH=7-7.5) with lipase (Candida cylindracea) at 55° C.-65° C., preferably at 60° C. for 70-80 hrs to yield a single enantiomer of formula 23 and leaving the acetate of formula 22 enriched in the other enantiomer (>95% based on NMR);
(2) treating alcohol of formula 23 as obtained in step (1) with a mixture of ammonia and sodium in THF/EtOH to yield product 24;
(3) treating compound of formula 24 as obtained in step (2) with benzoyl chloride to obtain compound 25,
(4) ketal hydrolysis of compound 25 as obtained in step (3) to give lactone 26;
(5) oxidizing lactone 26 as obtained in step 4 with mCPBA to yield an inseparable mixture of 27 and 28;
(6) reducing the above mixture 27 and 28 as obtained in step (5) to yield an inseparable mixture of 29 and 30 along with product 31;
(7) treating mixture of 29 and 30 as obtained in step (6) with benzaldehydedimethylacetal to yield the benzylidene derivative 32 from 29, leaving triol 30 unreacted;
(8) converting Triol 30 as obtained in step (7) to its triacetate derivative 33 for characterization;
(9) converting Stereochemically pure 30 as obtained in step (7) to the corresponding DMTr-protected base-containing phosphoramidite X by standard procedures.

Novel synthesized compounds were characterized by ¹H, ¹³C and ³¹P NMR and mass spectral analysis.

The synthesized phosphoramidites were used in the synthesis of 2′-5′-linked 3′-deoxyoligomers by application on an automated solid-phase DNA synthesizer. The length of the entire oligomer is 10 to 30 nucleoside units, preferably 18 nucleoside. The resulting oligomers were purified by reverse-phase HPLC on a C18 column and characterized my MALDI-TOF spectrometry.

The phosphoramidite nucleosides of present invention are incorporated in 2′-5′-linked ribo/deoxyribonucleic acid oligomers and converted to duplexes with 3′-5′RNA.

The 2′-5′-linked nucleic acid oligomers of present invention comprises a length of 10-30 nucleoside units having 1 or more phosphoramidite nucleoside monomer units.

The 3′-deoxy,2′-5′-linked oligomers synthesized using the S-type locked uridine phosphoramidites are listed below in Table 1.

TABLE 1
2′-3′-5′-linked oligomers synthesized using
S-type locked uridine phosphoramidite (IV)

| Sequence | Sequence (5′ → 2′) | Length |
|---|---|---|
| 2′-DNA-S-1s | CAC CAT TGT CAC AC [symbol] CCA | 18 |
| 2′-DNA-S-1d | CAC CAT TG [symbol] CAC AC [symbol] CCA | 18 |
| 2′-DNA-S-1t | CAC CA [symbol] TG [symbol] CAC AC [symbol] CCA | 18 |
| 2′-DNA-S-2s | CCT CTT ACC TCA GT [symbol] ACA | 18 |
| 2′-DNA-S-2d | CCT CTT ACC [symbol] CA GT [symbol] ACA | 18 |
| | 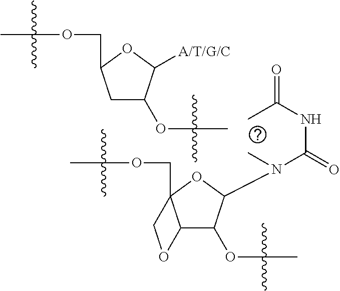 | |
| 2′-DNA-S-2t | CCT CT [symbol] ACC [symbol] CA GT [symbol] ACA | 18 |

[symbol]

= locked S-type nucleoside monomer
A/T/G/C =

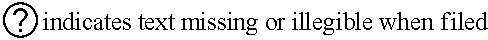

Similarly, 3′-deoxy, 2′-5′-linked oligomers were synthesized using the N-type locked uridine phosphoramidites and are listed below in Table 2.

TABLE 2
2′-5′-linked oligomers synthesized using
N-type locked uridine phosphoramidite (II)

| Sequence | Sequence (5′ → 2′) | Length |
|---|---|---|
| 2′-DNA-N-1s | CAC CAT TGT CAC AC [symbol] CCA | 18 |
| 2′-DNA-N-1d | CAC CAT TG [symbol] CAC AC [symbol] CCA | 18 |
| 2′-DNA-N-1t | CAC CA [symbol] TG [symbol] CAC AC [symbol] CCA | 18 |
| | 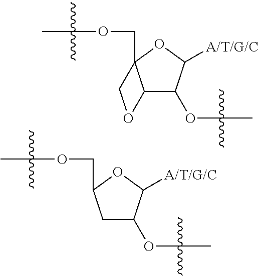 | |
| 2′-DNA-N-2s | CCT CTT ACC TCA GT [symbol] ACA | 18 |
| 2′-DNA-N-2d | CCT CTT ACC [symbol] CA GT [symbol] ACA | 18 |
| 2′-DNA-N-2t | CCT CT [symbol] ACC [symbol] CA GT [symbol] ACA | 18 |

[symbol]

= locked N-type nucleoside monomer
A/T/G/C =

The 5′-O-(4,4′-dimethoxytrityl)-β-D-xylofuranosyl 3′-deoxy-3′-fluoro-uridinyl-2′-O-phosphoramidite (VIIIb) was used in the synthesis of 3′-deoxy-2′-5′-linked oligomers listed in Table 3.

TABLE 3
3′-deoxy-2′-5′-linked oligomers
using 5′-O-(4,4′-dimethoxytrityl)-β-
D-xylofuranosyl 3′-deoxy-3′-
fluoro-uridinyl-2′-O-phosphoramidite (VIIIb)

| Sequence | Sequence (5′ → 2′) | Length |
|---|---|---|
| 2′-DNA-uxf-1s | CAC CAT TGT CAC AC [symbol] CCA | 18 |
| | 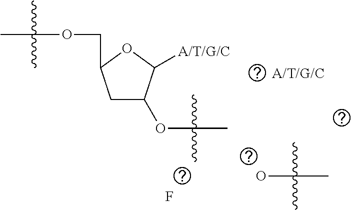 | |
| 2′-DNA-uxf-1d | CAC CAT TG [symbol] CAC AC [symbol] CCA | 18 |

[symbol]

= fluoro xylonucleoside monomer
A/T/G/C =

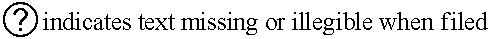

The 5′-O-(4,4′-dimethoxytrityl)-β-D-ribofuranosyl 3′-deoxy-3′-fluoro-uridinyl-2-O-phosphoramidite (VIIIa) was used in the synthesis of 3′-deoxy-2′-5′-linked oligomers listed in Table 4.

TABLE 4
3′-deoxy-2′-5′-linked oligomers using 5′-
O-(4,4′-dimethoxytrityl)-β-
D-ribofuranosyl 3′-deoxy-3′-fluoro-
uridinyl-2-O-phosphoramidite (VIIIa)

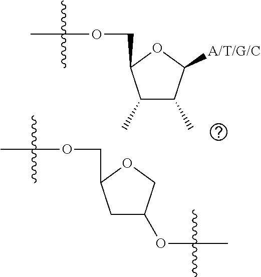

| Sequence | Sequence (5′ → 2′) | Length |
|---|---|---|
| 2′-DNA-urf-1s | CAC CAT TGT CAC AC [symbol] CCA | 18 |
| 2′-DNA-urf-1d | CAC CAT TG [symbol] CAC AC [symbol] CCA | 18 |

[symbol]

= fluoro ribonucleoside monomer
A/T/G/C =

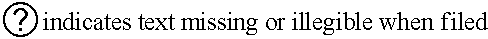

The control 3′-deoxy-2′-5′-linked oligomers were also synthesized for comparative studies and are listed in Table 5.

TABLE 5
3′-deoxy-2′-5′-linked oligomers synthesized

| | | |
|---|---|---|
| 2′-DNA-1 | CAC CAT TGT CAC ACT CCA | 18 |
| 2′-DNA-2 | CCT CTT ACC TCA GTT ACA | 18 |

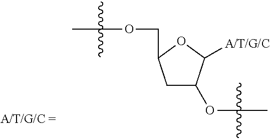

The synthesized oligomers along with their MALDI-TOF spectrometric data are listed in Table 6.

TABLE 6
MALDI-TOF spectrometric data of synthesized
3′-deoxy-2′-5′-linked oligomers.

| | | Mass | |
|---|---|---|---|
| Entry | Sequence | Calc. | Obs. |
| 2′DNA-1 | CACCATTGTCACACTCCA | 5363 | 5362 |
| 2′DNA-N-1s | CACCATTGTCACAC [symbol] CCA | 5377 | 5380 |
| 2′DNA-N-1d | CACCATTG [symbol] CACAC [symbol] CCA | 5391 | 5393 |
| 2′DNA-N-1t | CACCA [symbol] TG [symbol] CACAC [symbol] CCA | 5405 | 5405 |
| 2′DNA-S-1s | CACCATTGTCACAC [symbol] CCA | 5377 | 5376 |
| 2′DNA-S-1d | CACCATTG [symbol] CACAC [symbol] CCA | 5391 | 5391 |
| 2′DNA-S-1t | CACCA [symbol] TG [symbol] CACAC [symbol] CCA | 5405 | 5404 |
| 2′DNA-2 | CCTCTTACCTCAGTTACA | 5369 | 5368 |
| 2′DNA-N-2s | CCTCTTACCTCAGT [symbol] ACA | 5383 | 5386 |
| 2′DNA-N-2d | CCTCTTACC [symbol] CAGT [symbol] ACA | 5397 | 5401 |
| 2′DNA-N-2t | CCTCT [symbol] ACC [symbol] CAGT [symbol] ACA | 5411 | 5409 |
| 2′DNA-S-2s | CCTCTTACCTCAGT [symbol] ACA | 5383 | 5389 |
| 2′DNA-S-2d | CCTCTTACC [symbol] CAGT [symbol] ACA | 5397 | 5401 |
| 2′DNA-S-2t | CCTCT [symbol] ACC [symbol] CAGT [symbol] ACA | 5411 | 5409 |
| 2′DNA-uxf-1s | 5′CAC CAT TGT CAC ACU^{xF} | 5368 | 5368 |
| | CCA2′ | | |
| 2′DNA-uxf-1d | 5′CAC CAT TG [symbol] CAC ACU^{xF} | 5372 | 5372 |
| | CCA2′ | | |
| 2′DNA-urf-1s | 5′CAC CAT TGT CAC ACU^{rF} | 5368 | 5365 |
| | CCA2′ | | |
| 2′DNA-urf-1d | 5′CAC CAT TG [symbol] CAC ACU^{rF} | 5372 | ....* |
| | CCA2′ | | |

*data awaited

The synthesized oligomers bearing the N-type and S-type locked uridine units were assessed for their ability to bind to complementary DNA and RNA by UV-melting experiments. The results are delineated in Table 7.

TABLE 7
UV-melting data for DNA:isoDNA and RNA:isoDNA
duplexes^{a}

| | | | ΔTₘ | | | | ΔTₘ |
|---|---|---|---|---|---|---|---|
| Code No. | Tₘ (° C.) | | (° C.) | Code No. | Tₘ (° C.) | | (° C.) |
| | DNA3 | RNA3 | | | DNA3 | RNA3 | |
| 2′DNA-1 | ND | 50.5 | − | 2′DNA-1 | ND | 50.5 | − |
| 2′DNA-N- | ND | 52.2 | +1.6 | 2′DNA-S-1s | ND | 51.0 | +0.5 |
| 1s | | | | | | | |
| 2′DNA-N- | ND | 42.9 | −7.6 | 2′DNA-S-1d | ND | 52.8 | +2.3 |
| 1d | | | | | | | |
| 2′DNA | ND | 42.3 | −8.2 | 2′DNA-S-1t | ND | 50.5 | 0.0 |
| | DNA4 | RNA4 | | | DNA4 | RNA4 | |
| 2′DNA-2 | ND | 46.0 | − | 2′DNA-2 | ND | 46.0 | − |
| 2′DNA-N- | ND | 46.4 | +0.3 | 2′DNA-S-2s | ND | 47.0 | +1.0 |
| 2s | | | | | | | |
| 2′DNA-N- | ND | 41.9 | −4.1 | 2′DNA-S-2d | ND | 46.7 | +0.6 |
| 2d | | | | | | | |
| 2′DNA-N- | ND | 41.9 | −4.1 | 2′DNA-S-2t | ND | 47.2 | +1.1 |
| 2t | | | | | | | |

DNA3: 5′-TGGAGTGTGACAATGGTG-3′,
RNA3: 5′-UGGAGUGUGACAAUGGUG-3,
DNA4: 5′-TGTAACTGAGGTAAGAGG-3′,
RNA4: 5′-UGUAACUGAGGUAAGAGG-3′

Melting temperatures were obtained from the maxima of the first derivative of the melting curves (A_{260 nm} vs. temperature) in a buffer containing 10 mM sodium phosphate, 150 mM sodium chloride, pH 7.0 using 1.0 μM concentration of each strand. Tₘ values were averaged over at least three measurements and are accurate to ±0.5° C. Tₘs of unmodified duplexes are shown in bold. ND=not detected (non-binding).

The oligomers bearing the 3′-xylofluoro-uridine and 3′-ribofluoro-uridine units were assessed for their ability to bind to complementary DNA and RNA by UV-melting experiments. The results are delineated in Table 8.

TABLE 8
UV-melting data for DNA:isoDNA and RNA:isoDNA
duplexes^{a}

| | Tₘ (° C.) | | ΔTₘ | | Tₘ (° C.) | | ΔTₘ |
|---|---|---|---|---|---|---|---|
| Code No. | DNA3 | RNA3 | (° C.) | Code No. | DNA3 | RNA3 | (° C.) |
| 2′DNA-1 | ND | 50.5 | − | 2′DNA-1 | ND | 50.5 | − |
| 2′DNA- | ND | 45.7 | −4.8 | 2′DNA- | ND | 49.0 | −1.5 |
| uxf-1s | | | | uxf-1s | | | |
| 2′DNA- | ND | 45.2 | −5.3 | 2′DNA- | ND | | ....* |
| uxf-1d | | | | urf-1d | | | |

*data awaited
DNA3: 5′-TGGAGTGTGACAATGGTG-3′,
RNA3: 5′-UGGAGUGUGACAAUGGUG-3,

Melting temperatures were obtained from the maxima of the first derivative of the melting curves (A_{260 nm} vs. temperature) in a buffer containing 10 mM sodium phosphate, 150 mM sodium chloride, pH 7.0 using 1.0 μM concentration of each strand. Tₘ values were averaged over at least three measurements and are accurate to ±0.5° C. Tₘs of unmodified duplexes are shown in bold. ND=not detected (non-binding).

The 2′-5′ RNA backbone is stable and is known to form weak complexes with target RNA. The conformational constraint in the 2′-5′ backbone in the form of locked S-type or locked N-type monomer or 3′-fluoro-3′deoxy-xylo/ribo unit or cyclohexenyl monomer unit could enhance the strength of complexes of 2′-5′-linked oligomers with 3′-5′ RNA. Thus, all 2′-5′-linked oligomers synthesized were found to bind selectively only to complementary RNA, while no binding was observe to the complementary DNA sequences. The complexes formed with oligomers containing increasing number of N-type locked monomer with complementary RNA were destabilized compared to the control 2′-5′-sequence (ΔTₘ≈−4.1 to −8.2° C.). In contrast, the oligomers containing the S-type locked monomers slightly stabilized the complexes with RNA (ΔTₘ≈+0.5 to +2.0° C.). Oligomers bearing three S-type modified units also formed complexes with RNA, although the effect was not found to be additive. Similarly, the duplexes formed with oligomers containing 3′-fluoro-xylofuranosyl uridine were destabilized (ΔTₘ≈−4.8 to −5.3° C.) in comparison to the control duplex, while the duplex formed with oligomers containing 3′-fluoro-ribofuranosyl uridine showed a stability similar to the control duplex (only minimal destabilization, ΔTₘ=−1.5° C.).

The consequent incorporation of the four synthesized monomers into 2′-5′ linked oligomers and their biophysical implications on the stability of the said duplexes has explicit importance towards development into therapeutic oligomers. The intrinsic stability of 2′-5′ phosphodiester linkage as opposed to 3′-5′ linked oligomers (which are susceptible to enzymatic cleavage) is also an added advantage. Conceptually, these are new molecular entities for antisense, siRNA based drug development.

The antisense molecule (which block translation in vivo of specific mRNAs, thereby preventing the synthesis of protein which are undesired or harmful to the cell/organism) or siRNA (small interfering RNA) prepared using the above synthesized molecular entities can be used for formulating drug by incorporating customary auxillary such as buffers/stabilizers/pharmaceutical acceptable carrier.

The described compounds and the route of syntheses as described herein can be varied with regard to groups attached to the locked nucleosides, the positions of the groups, the starting compounds for the syntheses, the process conditions, the intermediates and such like.

The following examples, which include preferred embodiments, will serve to illustrate the practice of this invention, it being understood that the particulars shown are by way of example and for purpose of illustrative discussion of preferred embodiments of the invention. It should be understood that the invention is not to be limited to the specific conditions or details described in the following examples.

### EXAMPLE 1

### Process for preparing 2′-O-phosphoramidite of N-type locked uridine

### (i) Preparation of 5-O-allyloxycarbonyl-3,6-anhydro-1,2-isopropylidene-Glucofuranose (6)

3,6-anhydro-1,2-isopropylidene-5-hydroxy-Glucofuranose (5) (0.460 g, 2.28 mmol) was dissolved in dry dichloromethane (9 ml). Anhydrous pyridine (0.38 ml) was added and the reaction mixture was cooled to 0° C. in an icebath. Allyloxycarbonyl chloride (0.3 ml; 2.91 mmol) was added dropwise and then the reaction was stirred at room temperature for two hrs, when TLC showed absence of starting compound. Reaction mixture was extracted with dichloromethane, followed by water wash and drying over sodium sulphate. Removal of solvent yielded a sticky gum having compound (6), which was used without any further purification.

Yield: 0.650 g, 99%.

¹H NMR (CDCl₃, 200 MHz): δ 1.34 (s, 3H, CH₃), 1.49 (s,3H, CH₃), 3.78-3.86 (m, 1H, H6), 4.00-4.08 (m, 1H, H6′), 4.54-4.56 (d, 1H, H5), 4.61-4.67 (m, 3H, H2,H3,H4), 4.99-5.09 (m, 2H, allylic-H), 5.25-5.43 (m, 2H, allylic-H), 5.84-6.03 (bm, 1H, allylic-H) 6.00 (d, 1H, H1, J=3.55 Hz).

“C NMR (CDCl₃, 50.32 MHz): δ 26.72 (CH₃), 27.34 (CH₃), 66.78 (CH₂, C6), 68.87 (O-CH₂-allyl) 76.05 (CH, C2), 80.70 (CH, C3), 84.80 (CH, C4), 85.25 (C5) 107.18 (CH, Cl), 112.78 (C), 119.13 (CH₂═CH-allyl), 131.10 (CH-allyl), 154.14 (C).

### (ii) Preparation of 1,2-di-O-acetyl-3,6-anhydro-5-O-allyloxycarbonyl-α,β-Glucofuranose (7)

Compound (6) obtained from step (i) was desiccated (1.5 g, 0.524 mmol) and dissolved in acetic acid (16 ml). Acetic anhydride (1.6 ml) was added, after cooling the reaction flask to 10° C., followed by dropwise and slow addition of concentrated sulfuric acid (0.16 ml). Reaction mixture was stirred overnight at room temperature. TLC indicated complete product formation. The reaction was quenched with ice and 5% aqueous NaHCO₃, then extracted with dichloromethane, followed by water wash and drying over sodium sulphate. After solvent removal the crude product was purified on a silica gel column using petroleum ether and ethyl acetate as an eluant. The compound 7 was eluted in 30% ethyl acetate in petroleum ether.

Yield: 1.32 g, 76.3%

¹HNMR (CDCl₃, 200 MHz): δ 2.07 (s, 3H, CH₃), 2.09 (s,3H, CH₃), 3.84-3.92 (m, 1H, H6), 4.05-4.11 (m, 1H, H6′), 4.64-4.67 (m, 2H, H3,H4), 4.75-4.80 (m, 1H, H2), 4.95-5.0 (m, 2H, allyl-H), 5.26-5.41 (m, 2H, allyl-H), 5.84-6.23 (m, 1H, allyl-H), 6.52 (d, 1H, H1,J=4.2 Hz).

¹³C NMR (CDCl₃, 50.32 MHz): δ 20.32 (CH₃), 20.75 (CH₃), 68.44 (CH₂, C6), 68.5.4 (O—CH₂-allyl) 74.97 (CH, C2), 80.39 (CH, C3), 83.09 (CH, C4), 85.21 (C5) 100.25 (CH, Cl), 118.74 (CH₂═CH-allyl), 131.04 (CH, allyl), 153.88 (C═O, allyl), 169.07 (2C═O, acetate).

IR (CHCl₃): vₘₐₓ 3024, 1749, 1650, 1428.16, 1371.84 1266.85 1114.45, 1061.37, 1024.79, 957.51, 879.82, 755.49, 667.5,599.59.

### (iii) Preparation of 2′-O-acetyl-3′,6′-anhydro-5′-O-allyloxycarbonyl-Uridine (8):

Compound (7) (1.110 g, 3.36 mmol), obtained from the previous step was dissolved in anhydrous acetonitrile (35 ml). Reaction flask was flushed with nitrogen and uracil (0.452 g, 4.032 mmol) was added. N,O-Bis(trimethylsilyl)acetamide (BSA) (0.27 ml, 1.1 mmol) was added to the reaction flask under nitrogen atmosphere. Then the reaction mixture was refluxed at 70° C. for one hour cooling in an ice bath. TMSOTf (0.23 ml, 1.27 mmol) was added slowly with a syringe and the reaction mixture was refluxed for three hours. TLC showed disappearance of starting material and appearance of a lower moving UV positive spot which charred on acid spray and heat. The reaction mixture was cooled to room temperature, diluted with dichloromethane, washed with NaHCO₃ and water, dried over sodium sulphate followed by solvent removal. The crude product was purified on a silica gel column using dichloromethane and methanol as an eluant. Compound (8) eluted in 2.5% methanol in dichloromethane.

Yield: 1.19 g, 92.96%.

¹HNMR (CDCl₃, 200 MHz): δ 2.13 (s, 3H, CH₃), 4.09-4.12 (m, 2H, H6′, H6″), 4.54-4.57 (m, 1H, H4′), 4.62-4.66 (m, 2H, H3′, H2′), 4.93-4.98 (m, 1H, H5′), 5.15-5.41 (bm, 4H, allyl-H), 5.78-5.83 (d, 1H, H5) 5.84-6.01 (m, 1H, allyl-H), 6.24 (d, 1H, H1′, J=3.88 Hz), 7.62 (d, 1H, H6), 8.84 (bs, 1H, NH).

¹³C NMR (CDCl₃, 50.32 MHz): δ 20.45 (CH₃), 69.06 (CH₂, C6′), 70.99 (O—CH₂-allyl), 76.00 (CH, C2′), 79.26 (CH, C4′), 81.22 (CH, C3′), 85.50 (CH, C5′), 90.59 (CH, C5-Uridine), 103.40 (CH, C1′), 119.47, (CH₂═CH, allyl), 130.81 (CH, allyl), 139.78 (CH, C6-Uridine), 150.34 (C═O, C4-Uridine), 153.97 (C═O, allyl), 163.11 (C═O,C2-Uridine),169.49 (C═O, acetate).

IR (CHCl₃): vₘₐₓ 3391.85, 3020.46, 2924.68, 2853.76, 1751.23, 1694.89, 1458.89, 1381.57, 1265.47, 1216.15, 1090.08, 1060.10, 758.86, 668.59.

Mass (LCMS): m/z 383.05 M+1, 405.06 M+23(Na)

### (iv) Preparation of 5′-hydroxy-2′-O-acetyl-3′,6′-anhydro-Uridine (9)

Compound (8) (1.19 g, 3.14 mmol) obtained in the previous step, was dissolved in dichloromethane (45 ml). PPh₃ (0.540 g, 2 equivalents) was added followed by piperidine (2.0 ml) and Tris(dibenzylidene acetone) dipalladium [Pd₂(dba)₃] (0.150 g). The reaction mixture was stirred for 10 minutes. TLC showed absence of starting compound. Solvent was removed and the crude product was given a wash with solvent ether. Column purification done on a silicagel column, pure compound (9) eluted in methanol (3.5%) in dichloromethane.

Yield: 0.650 g, 70%

¹H NMR (CDCl₃, 500 MHz): δ 2.13 (s, 3H, CH₃), 2.75 (d, 1H, OH), 3.85-3.88 (m, 1H, H6″), 4.04-4.08,(m, 1H, H6′), 4.51-4.52 (m, 2H, H4′, H5′),4.71-4.72 (m, 1H, H3′), 5.28-5.29 (m, 1H, H2′) 5.82 (d, 1H, H5, J=8.19 Hz), 6.23 (d, 1H, H1′, J=3.6 Hz), 7.59 (d, 1H, H6, J=8.32 Hz), 8.45 (s, 1H, NH).

¹³C NMR (CDCl₃, 50.32 MHz): δ 20.55 (CH₃), 72.34 (CH, C2′) 73.82 (CH₂, C6′), 79.82(CH, C4′), 82.77(CH, C3′), 85.53 (CH, C5′), 91.30 (CH, C5-Uridine), 103.85(CH, C1′), 140.45 (CH, C6-Uridine), 150.03 (C=O, C4-Uridine), 162.42 (C═O, C2-Uridine), 169.58 (C═O, acetate).

Mass (LCMS): m/z 299.02 M+1, 321 M+23(Na).

### (v) Preparation of 5′-O-dimethoxytrityl-2′-O-acetyl-3′,6′-anhydro-Uridine (10)

The substrate (9), (0.430 g, 1.443 mmol) was coevaporated with anhydrous pyridine twice, then dissolved in pyridine (10 ml). 4,4′-dimethoxytritylchloride (1.467 g, 4.329 mmol, 3 equivalents) was added in one lot. Reaction mixture was stirred overnight at room temperature. TLC done showed a faster moving trityl positive spot which charred on acid spray and heat. The reaction was quenched with methanol, extracted with dichloromethane, washed with NaHCO₃ and water, dried over sodium sulphate, followed by solvent removal. The crude product was purified on a silica gel column using dichloromethane, methanol and pyridine (0.5%) as an eluant. Compound (10) obtained is eluted in methanol (1.5%) in dichloromethane.

Yield: 0.730 g, 84.39%.

¹H NMR (CDCl₃, 200 MHz): δ 2.09 (s, 3H, CH₃), 3.22-3.44 (m, 2H, H6′, H6″), 3.8 (s, 6H, OCH₃), 4.13-4.30 (m, 3H, H4′, H3′, H2′), 5.14-5.16 (m, 1H, H5′), 5.79 (d, 1H, H5, J=8.10 Hz), 6.05 (d, 1H, H1′, J=3.4 Hz), 6.82-6.87 (m, 4H, Dmtr.), 7.31-7.5 (m, 9H, Dmtr.),7.81 (d, 1H, H6, J=8.17 Hz), 8.65 (s, 1H, NH).

### (vi) Preparation of 5′-O-dimethoxytrityl-2′-hydroxy-3′,6′-anhydro-Uridine (11)

The substrate (10) (0.700 g, 1.16 mmol) was dissolved in AR grade methanol (50 ml). Aqueous ammonia (15 ml) was added and the pinkish slightly turbid reaction mixture was stirred for one hour at room temperature. TLC showed the absence of starting compound. Solvent was removed to get a yellowish solid. The solid was redissolved in dichloromethane and given water wash. The organic layer was dried over sodium sulfate and concentrated to get pale yellow solid foam. Purification was done on a silica gel column using dichloromethane, methanol and pyridine (0.5%) as an eluant. Compound (11) eluted in methanol (2.5%) in dichloromethane.

Yield: 0.620 g, 93.23%.

¹H NMR (CDCl₃, 400 MHz): δ 3.21-3.31 (m, 2H, H6′, H6″), 3.8 (s, 6H, OCH₃), 4.16-4.17 (d, 1H, H4′), 4.34-4.38 (m, 1H, H3′), 4.44-4.47 (m,2H, H2′, H5′), 5.72 (d, 1H, H5, J=8.1 Hz), 5.79 (s, 1H, H1′), 6.83-6.86 (m,4H, Dmtr.), 7.21-7.41 (m, 9H, Dmtr.) 8.83 (d,1H, H6, J=8.1 Hz), 8.62 (s, 1H, NH), 10.41 (bs, 1H, OH)

¹³C NMR (CDCl₃, 100.61 MHz): 55.26 (OCH₃), 72.27 (CH₂, C6′), 75.10 (CH, C4′), 80.11 (CH, C3′), 85.38 (CH, C5′), 86.78 (CH, C2′), 88.01 (C), 96.02 (CH, C5-Uridine), 101.34 (CH, Cl), 113.38-113.41 (2CH), 123.82 (2CH), 127.18 (CH), 127.86 (CH), 128.15 (CH), 129.95 (2CH), 135.87 (C), 136.01 (C), 136.09 (CH), 140.62 (CH, C6-Uridine), 144.94 (C), 149.66 (3CH), 150.89 (C, C4-Uridine), 158.84 (2C), 164.24 (C, C2-Uridine)

### (vii) Preparation of 5′-O-Dimethoxytrityl-3-O,5′-C-methylene (uridine) xylonucleoside-2′-O-phosphoramidite (II)

Compound (11) (0.200 g, 0.358 mmol) was co-evaporated with dry dichloromethane, and then dissolved in dichloromethane (3.0 ml). Diisopropylethylamine (DIPEA) (0.180 ml, 0.954 mmol) was added, followed by chloro(2-cyanoethoxy)-N,N-diisopropyl amino)-phosphine (0.153 ml, 0.661 mmol) at 0° C. The reaction mixture was stirred under argon atmosphere at room temperature for 3 hours. TLC done indicated the absence of starting material. The reaction mass was diluted with dichloromethane, washed with NaHCO₃and water, dried over sodium sulphate, followed by solvent removal. The crude product was purified on a silica gel column using 1:1 mixture of dichloromethane:ethylacetate and 1% triethylamine.

Yield: 0.160 g, 58.9%.

³¹P NMR (CDCl₃): δ 150.72, 152.14.

### EXAMPLE 2

### Process for preparing 2′-O-phosphoramidite of S-type locked uridine wherein n=1:

### (i) Preparation of 2′-3′-O-cyclohexylidene-uridine

To a mixture of uridine (5.0 g, 20.492 mmol) and pTSA (0.352 g, 2.0492 mmol), was added cyclohexanone (30 ml). The reaction was stirred overnight at r.t. It was warmed at 40-50° C. for 3 h, when a clear solution was obtained and TLC indicated consumption of starting material and appearance of a faster-moving spot. Addition of petroleum ether resulted in the precipitation of a white solid, which was filtered off and washed thoroughly with petroleum ether. It was then dried by desiccation to afford the desired product in quantitative yield (6.99 g).

¹H NMR (CDCl₃) δ: 9.88 (s, 1H, N3H), 7.46 (d, J=8.09 Hz, 1H, H6), 5.74 (d, J=8.09 Hz, 1H, H5), 5.65 (d, J_{1′2}′=2.65 Hz, 1H, H1′), 5.01 (dd, J=2.78,6.31 Hz, 1H, H2′), 4.93 (dd, J=3.29, 6.32 Hz, 1H, H3′), 4.29 (m, 1H, H4′), 3.86 (m, 2H, H5′, H5″), 3.48 (br, 1H, OH), 1.75, 1.58, 1.40 (m, 10H, cyclohexyl CH₂×5).

¹³C NMR (CDCl₃) δ: 163.9 (C2), 150.5 (C4), 143.1 (C6), 115.1 (O2′-C—O3′), 102.5 (C5), 95.3 (C1′), 87.1 (C2′), 83.3 (C3′), 79.9 (C4′), 62.4 (C5′), 37.0, 34.6, 24.8, 23.9 and 23.5 (cyclohexyl CH₂×5).

### (ii) Preparation of 5′-aldehydo-2′,3′-O-cyclohexylidene-uridine

2′,3′-O-cyclohexylidene-uridine (200 mg, 0.617 mmol) was dissolved in acetonitrile (20.0 ml). To this, 2-Iodoxybenzoic acid (IBX) (519 mg, 1.852 mmol) was added and the mixture was heated at 80° C. for 2.5 h, when TLC examination revealed absence of starting material. The reaction was allowed to cool to r.t. and then, cooled on ice, before filtering off the IBX through a pad of Celite. The filtrate was concentrated, adsorbed on silica gel and immediately purified by short column chromatography on silica gel (60-120 mesh). Elution was carried out from 10 to 50% acetone/petroleum ether. The pure fractions were concentrated to give a solid white foam (160 mg, 76.5%), which was revealed to be the aldehyde hydrate by NMR.

¹H NMR (CDCl₃) δ: 9.44 (s, 1H, N3H), 7.30 (d, J=7.95 Hz, 1H, H6), 5.78 (d, J=7.83 Hz, 1H, H5), 5.50 (s, 1H, H1′), 5.22 (dd, J=6.19, 1.39, 1.52 Hz, 111, H4′?), 5.09 (d, J=6.32 Hz, 1H, H3′), 4.57 (d, J=1.39 Hz), 2.01 (br, 2H, OH×2), 1.72, 1.58, 1.41 (m, 10H, CH₂×5).

### (iii) Preparation of 4′-hydroxymethyl-2′,3′-O-cyclohexylidene-uridine

Ref: Jones, G. H.; Taniguchi, M.; Tegg, D.; Moffatt, J. G. J. Org. Chem. 1979, 44 (8), 1309-1317.

To a solution of 5′-aldehydo-2′,3′-O-cyclohexylidene-uridine (150 mg, 0.466 mmol) in dioxane (1.0 ml), was added aqueous formaldehyde (37%, 0.088 ml, 0.88 mmol), while cooling in a water bath. This was followed by addition of aqueous NaOH (2N, 0.44 ml, 0.88 mmol). After 5 min, the reaction was cooled in an ice-bath and NaBH₄ (32 mg, 0.839 mmol) was added. The clear yellow solution developed turbidity, but stayed yellow. Stirring was continued in an ice-bath for 15 min and then, at ˜20° C. (solution turned clear) for 2 h. TLC examination revealed the consumption of starting material and appearance of a lower-moving spot. The reaction was neutralized using Tulsion H⁺ resin. (According to the literature report, this neutralization was carried out by HCl). A small amount of white solid was formed, which did not dissolve in dioxane, water or ethanol. The resin and white solid were filtered off and washed with dioxane and ethanol. The filtrate was concentrated, silica gel was added to adsorb, and the mixture was immediately purified by column chromatography on silica gel (60-120 mesh). Elution was carried out from 5 to 10% MeOH in CHCl₃. The fractions of pure product were concentrated under vacuum to get a white solid (60 mg, 38.3%).

¹H NMR (DMSO-d₆) δ: 11.37 (br s, 1H, NH), 7.92 (d, J=8.08 Hz, 1H, H6), 5.90 (d, J=4.04 Hz, 1H, H1′), 5.66 (d, J=8.08 Hz, 1H, H5), 5.19 (t, J=4.93, 4.80 Hz, 1H, 2′-OH), 4.87 (t, J^{1′,2}=4.17, J_{2′,3}=6.06 Hz, 1H, H2′), 4.76 (d, J_{2′3}=6.07 Hz, 1H, H3′), 4.66 (t, J=5.56, 5.81 Hz, 1H, 3′-OH),3.55, 3.63 (m, 4H, CH₂OH×2), 1.67, 1.51, 1.36 (m, 10 H, CH₂×5).

¹³C NMR (DMSO-d₆) δ: 163.1 (C2), 150.4 (C4), 141.2 (C6), 113.3 (O2′-C-O3′), 101.8 (C5), 89.6 (C1′), 88.9 (C4′), 83.5 (C2′), 81.1 (C3′), 62.7 (CH₂OH), 60.7 (CH₂OH), 36.1, 34.0, 24.4, 23.5 and 23.1 (cyclohexyl CH₂×5).

### (iv) Preparation of 4′-p-toluenesulphonyl-methyl-2′,3′-O-cyclohexylidene-uridine

Ref: Obika, S.; Morio, K-i. M.; Nanbu, D.; Imanishi, T. Chem. Commun. 1997, 1643-1644.

p-Toluenesulphonyl chloride (458 mg, 2.401 mmol) was dissolved in anhydrous pyridine (3.0 ml) and added drop-wise to a stirred, ice-cooled solution of 4′-hydroxymethyl-2′,3′-O-cyclohexylidene-uridine (500 mg, 1.412 mmol) in anhydrous pyridine (10.0 ml). After 10 min, the reaction was heated at 110° C., 4.5 h. The reaction was then cooled to r.t., and satd. NaHCO₃ was added. The product was extracted in CH₂Cl₂ (×4). The organic layer was washed with brine, dried (Na₂SO₄) and concentrated to get a brown gum that was purified by silica gel column chromatography (100-200 mesh). Elution was carried out with 20 to 80% EtOAc in petroleum ether to get the pure product (310 mg, 43% yield). The un-reacted starting material could be recovered by elution with 1-3% MeOH in EtOAc.

¹H NMR (Acetone-d₆) δ: 10.21 (br s, 1H, NH), 7.84 (dd, J=7.46, 7.07 Hz, 2H, Ar, H6), 7.52 (d, J=7.09 Hz, 2H, Ar), 5.80 (d, J=3.66 Hz, 1H, H1′), 5.65 (d, J=8.08 Hz, 1H, H5), 5.09 (dd, J=3.67 Hz, 1H, H2′), 4.96 (d, J=6.32 Hz, 1H, H3′), 4.24 (ABq, J=10.36 Hz, 2H, CH₂OTs), 3.75 (ABq, J=11.25 Hz, 2H, CH₂OH), 2.49 (s, 3H, CH₃), 1.40-1.70 (m, 10H, CH₂×5).

¹³C NMR (Acetone-d₆) δ: 164.0 (C2), 151.3 (C4), 146.0 (Ar—C—CH₃), 142.9 (C6), 133.7 (Ar—SO₂—C), 130.8 (Ar—CH×2), 128.9 (Ar—CH×2), 115.4 (2′O-C—O3′), 102.9 (C5), 92.8 (C1′), 87.8 (C4′), 84.8 (C2′), 82.5 (C3′), 69.8 (CH₂OH), 36.9 34.8, 25.5, 24.6 and 24.2 (cyclohexyl CH₂×5), 21.6 (Ar—CH₃).

### (v) Preparation of 4′-p-toluenesulphonyl-methyl-uridine

Ref: Imanishi, T.; Obika, S. U.S. Pat. No. 6,770,748, 2004.

To 4′-p-toluenesulphonyl-4″-hydroxymethyl-2′,3′-O-cyclohexylidene-uridine (120 mg) was added de-ionized water (0.020 ml) and trifluoroacetic acid (0.98 ml). The reaction was stirred at r.t., 45 min. The TFA was evaporated under vacuum and co-evaporated with ethanol (×3). It was adsorbed on silica gel and purified by column chromatography (60-120 mesh). Elution was carried out from 0 to 20% MeOH in CHCl₃ to afford the product in 79% yield (80 mg). Unreacted starting material (20 mg) was recovered.

¹H NMR (Acetone-d₆) δ: 10.06 (br s, 1H, NH), 7.76 (overlapping d, J=8.33, 8.21 Hz, 3H, H6, Ar×2), 7.40 (d, J=7.96 Hz, 2H, Ar), 5.78 (d, J=6.69 Hz, 1H, H1′), 5.58 (dd, J=1.8, 8.21 Hz, 1H, H5), 4.37 (m, 2H, H2′, H3′), 4.23 (ABq, J=10.74 Hz, 2H, CH₂OH), 3.66 (s, 2H, CH₂OTs), 3.30 (br s, 3H, OH×3), 2.39 (s, 3H, Ar—CH₃).

¹³C NMR (Acetone-d₆) δ: 164.0 (C2), 151.7 (C4), 145.9 (Ar—C—CH₃), 141.8 (C6), 133.8 (SO₂—C), 130.8 (Ar—CH×2), 128.8 (Ar—CH×2), 103.1 (C5), 88.8 (C1′), 86.8 (C4′), 74.6 (C2′), 72.6 (C3′), 71.0 (CH₂OTs), 63.7 (CH₂OH), 21.6 (Ar—CH₃).

### (vi) (a) Preparation of 5′-dimethoxytrityl-4′-p-toluenesulphonyl-methyl-uridine

4′-p-Toluenesulphonylmethyl-uridine (120 mg, 0.299 mmol), dimethoxytrityl chloride (114 mg, 0.336 mmol), triethylamine (0.045 ml, 0.322 mmol) and DMAP (40 mg, 0.327 mmol) were stirred together in anhydrous pyridine (2.0 ml) overnight at r.t. Methanol (1.0 ml) was added and the solvents were evaporated and co-evaporated with dichloromethane under vacuum. The crude product was purified by silica gel (60-120 mesh) column chromatography. The silica gel was pre-inactivated with 1% Et3N. Elution was carried out from 50 to 100% EtOAc in petroleum ether,. followed by 1-3% MeOH in EtOAc, when the product was isolated upon concentration of the fractions. Unreacted starting material (50 mg) was recovered upon elution with 10% MeOH in EtOAc. (10 mg, 4.88% yield).

### (vi) (b) Improved preparation of 5′-dimethoxytrityl-4′-p-toluenesulphonyl-methyl-uridine using 5 equiv. DMTr-Cl

4′-p-Toluenesulphonylmethyl-uridine (100 mg, 0.234 mmol), dimethoxytrityl chloride (395 mg, 1.168 mmol) and DMAP (143 mg, 1.168 mmol) were stirred together in anhydrous pyridine (2.0 ml) at r.t., overnight. Methanol (1.0 ml) was added and the solvents were evaporated and co-evaporated with dichloromethane under vacuum. The crude product was purified by silica gel (60-120 mesh) column chromatography. The silica gel was pre-inactivated with 1% Et3N. Elution was carried out from 50 to 100% EtOAc in petroleum ether, followed by 1-3% MeOH in EtOAc, when the product was isolated upon concentration of the fractions. (70 mg, 41% yield).

¹H NMR (CDCl₃) δ: 10.33 (br s, 1H, NH), 7.75 (m, 2H, Ar), 7.36 (m, 3H, H6, Ar×2), 7.28 (m, 5H, DMTr), 7.15 (m, 4H, DMTr), 6.83 (d, 4H, DMTr), 5.80 (m, 2H, H1′, H5), 4.95 (m, 2H, OH×2), 4.29 (m, 1H, H2′), 3.80 (s, 6H, OCH₃×2), 3.65 (m, 3H, H3′, CH₂OTs), 3.39 (m, 2H, CH₂ODMTr), 2.85 (s, 3H, Ar—CH₃).

### (vii) Preparation of 5′-O-DMTr-O3′-4′-methylene bridged uridine

To 5′-dimethoxytrityl-4′-p-toluenesulphonyl-methyl-uridine (260 mg, 0.356 mmol), desiccated overnight, was added sodium hexamethyldisilazane (1.0M in THF, 3.56 ml, 3.562 mmol), while cooling in a water-bath (˜20° C.), under nitrogen atmosphere. The reaction was stirred 3 h. A satd. solution of NaHCO₃ was added and the solvents were evaporated under vacuum. The residue was was with ethylacetate and methanol and the washings, combined and evaporated to dryness. This was purified by column chromatography on silica gel (60-120 mesh). Elution was carried out using an increasing gradient of MeOH in ethylacetate. The pure product was obtained as a foamy solid in 25% yield (50 mg)

¹H NMR (CDCl₃) δ: 7.49 (d, J=8.21 Hz, 1H, H6), 7.32 (m, 2H, Ar), 7.27 (m, 7H, Ar), 6.84 (d, J=8.84 Hz, 4H, Ar), 6.46 (d, J=6.57 Hz, 1H, H1′), 5.56 (d, J=8.21 Hz, 1H, H5), 5.08 (d, J=8.21 Hz, 1H, H2′), 4.73,4.52 (ABq, J=8.09 Hz, 2H, 4′-CH₂—O-3′), 4.15 (m, 1H, H3′), 3.79 (s, 6H, OCH₃×2), 3.48 (m, 2H, H5′, H5″), 2.84 (s, 1H, OH).

### (viii) Preparation of 2′-O-phosphoramidite of 5′-O-DMTr-O3′,4-methylene-bridged uridine

To a solution of 5′-O-DMTr-O3′,4-methylene-bridged uridine (250 mg, 0.45 mmol) desiccated and dried by co-evaporation with anhydrous pyridine (2 mL×3), in anhydrous dichloromethane (2.0 mL), cooled in an ice-bath, was added N,N-diisopropylethylamine (0.4 ml, 2.24 mmol). This was followed by the addition of 2-cyanoethoxy-N,N-diisopropyl-chlorophosphine (0.15 ml, 0.67 mmol), and stirring was continued. After 2 h, when TLC indicated consumption of starting material, the reaction was worked up. It was diluted with dichloromethane. The organic layer was washed with a saturated solution of NaHCO3, brine and then dried over Na2SO4, and evaporated to dryness. The product was then purified by precipitation by dissolving in dichloromethane, followed by precipitation by hexane. This purification was repeated thrice to afford pure product in 91% yield (310 mg) as a foamy solid, which was further used in the solid phase synthesis of oligomers.

³¹P NMR (CDCl₃) δ: 150.6, 150.3.

### EXAMPLE 3

### Alternate method of preparing 5′-O-DMTr-O3′-4′-methylene bridged uridine

### (i) Preparation of O3′-4′-methylene-bridged uridine from 4′-p-Toluenesulphonylmethyl uridine

To a solution of 4′-p-Toluenesulphonylmethyl-uridine (150 mg, 0.350 mmol) in anhydrous toluene, was added sodium hexamethyldisilazane (1.0M in THF, 3.51 ml, 3.500 mmol), while cooling in a water-bath (˜20° C.), under nitrogen atmosphere. The reaction was stirred 3.5 h. A satd. solution of NaHCO₃ was added and the solvents were evaporated under vacuum. The residue was adsorbed on silica gel (60-120 mesh) and purified by column chromatography. Elution was carried out from 10 to 40% MeOH in dichloromethane. The pure product was obtained as a solid in quantitative yield (90 mg).

¹H NMR (DMSO-d₆) δ: 11.44 (br s, 1H, NH), 7.69 (d, J=8.08 Hz, 1H, H6), 6.26 (d, J=7.70 Hz, 1H, H1′), 5.72 (d, J=7.96 Hz, 1H, H5), 4.91 (d, J=4.42 Hz, 1H, H2′/3′), 4.76 (d, J=7.70 Hz, 1H, 4′-CH₂—O-3′), 4.32 (d, J=7.83 Hz, 1H, 4′-CH₂—O-3′), 4.01 (m, 1H, H2′/3′), 3.86 (m, 1H, H5′), 3.75 (d, J=5.31 Hz, 1H, H5″), 3.60 (d, J=5.81 Hz, 2H, OH×2).

### (ii) Preparation of 5′-O-DMTr-O3′-4′-methylene bridged uridine from O3′-4′-methylene-bridged uridine

1-(4-hydroxy-1-(hydroxymethyl)-2,6-dioxa-bicyclo[3.2.0]heptan-3-yl)pyrimidine-2,4(1H,3H)-dione (80 mg, 0.313 mmol) was co-evaporated with anhydrous pyridine (×3) and re-dissolved in anhydrous pyridine (0.5 ml). Dimethoxytrityl chloride (529 mg, 1.563 mmol) and DMAP (191 mg, 1.563 mmol) were added and the reaction was stirred at r.t., overnight. TLC examination showed appearance of a faster-moving spot, but starting material left largely unconsumed. However, the reaction was worked up. Methanol (1.0 ml) was added and the solvents were removed in vacuo. The residue was dried by co-evaporation with dichloromethane (×3), adsorbed on silica gel (60-120 mesh, pre-inactivated with 1% Et₃N) and purified by column chromatography. Elution was carried out on pre-inactivated silica gel using 50 to 100% EtOAc in petroleum ether, when the upper impurities were eluted. Subsequent elution with 2-3% MeOH in EtOAc yielded the pure product in 17.2% yield (30 mg). The unreacted starting material was recovered by elution with 20-30% MeOH in EtOAc.

¹H NMR (CDCl₃) δ: 7.49 (d, J=8.21 Hz, 1H, H6), 7.32 (m, 2H, Ar), 7.27 (m, 7H, Ar), 6.84 (d, J=8.84 Hz, 4H; Ar), 6.46 (d, J=6.57 Hz, 1H, H1′), 5.56 (d, J=8.21 Hz, 1H, H5), 5.08 (d, J=8.21 Hz, 1H, H2′), 4.73,4.52 (ABq, J=8.09 Hz, 2H, 4′-CH₂—O-3′), 4.15 (m, 1H, H3′), 3.79 (s, 6H, OCH₃×2), 3.48 (m, 2H, H5′, H5″), 2.84 (s, 1H, OH).

### EXAMPLE 4

### (i) Process for preparing 5-O-dimethoxytrityl-3′-deoxy-3′-fluoro-xylofuranosyl uridine

A mixture of 3′-deoxy-3′-fluoro-5′-hydroxy-xylofuranosyl uridine (500 mg), 4,4′-dimethoxytrityl chloride (0.6 g) and 4-dimethylaminopyridine in catalytic amount was dissolved in pyridine (5 ml). The reaction mixture was stirred at room temperature for 12 h. Pyridine was removed under vacuum. The residue dissolved in ethyl acetate (100 ml), washed with saturated NaHCO₃ (2×50 ml) and saturated aqueous NaCl (2×30 ml). The ethyl acetate layer was dried over Na₂SO₄, filtered and evaporated to dryness. The crude product was purified by silica gel (neutralized with Et₃N) column chromatography using EtOAC/pet ether (9:1) to get the title compound. (0.4 g , white foam).

Yield: 36%

¹H NMR (CDCl₃): δ: 7.46 (d, 1H, J_{5,6}=8.21 Hz, H6), 7.26-7.35(m, 9H DMT), 6.87(d, 4H, DMT) 5.81(s, 1H, H1′), 5.63 (d, 1H, J₆₅=8.21 Hz, H5), 4.86-5.10 (dd, 1H, J_{3′,F}=50.78 and J_{3′,4′}=3.16 Hz, H3′), 4.51-4.73 (m, 1H, H4′), 4.40 (d, 1H, H2′), 3.80 (s, 6H, 2×OMe), 3.53 (m, 2H, H5′,5″).

### (ii) Preparation of 5′-O-(4,4′-dimethoxytrityl)-β-D-xylofuranosyl-3′-deoxy-3′-fluoro-uridinyl -2-O-phosphoramidite (VIIIb)

Compound (21) (100 mg) obtained from previous step was dissolved in dry DCM (1 ml) followed by the addition of diisopropyl ethyl amine (40 μ) and chloro (2-cyanoethoxy)-(N,N-diisopropylamino)-phosphine (40 μl) and the reaction mixture was stirred at room temperature for 2 h. The contents were then diluted with dry DCM and washed with 5% NaHCO₃ solution. The organic phase was dried over anhydrous Na₂SO₄ and concentrated to foam. The residue was dissolved in DCM and precipitated with hexane to obtain compound of formula 26 (105 mg).

Yield: 80%

³¹P NMR (CDCl₃) δ: 153.6, 151.2.

### EXAMPLE 5

### (i) Process for preparing 5-O-dimethoxytrityl-3′-deoxy-3′-fluoro-ribofuranosyl undine (21)

A mixture of 3′-deoxy-3′-fluoro-5′-hydroxy-ribofuranosyl uridine (20) (1 mmol), 4,4′-dimethoxytrityl chloride (3 mmol) and 4-dimethylaminopyridine in catalytic amount was dissolved in pyridine (5 ml). The reaction mixture was stirred at room temperature for 2 h. Pyridine was removed under vacuum. The residue dissolved in ethyl acetate (100 ml), washed with saturated NaHCO₃ (2×50 ml) and saturated aqueous NaCl (2×30 ml). The ethyl acetate layer was dried over Na₂SO₄, filtered and evaporated to dryness. The crude product was purified by silica gel (neutralized with Et₃N) column chromatography using EtOAC/pet ether (9:1) to get the title compound (white foam).

Yield: 70%

¹H NMR (CDCl₃): δ:7.70-7.74(d, 1H, J_{5,6}=8.09 Hz, H6), 7.23-7.34 (m, 9H, DMT), 6.83-6.88 (d, 4H, DMT), 6.15-6.18 (d, 1H, H1′), 5.43-5.47 (d, 1H, J_{6,5}=8.09 Hz, H5), 4.94-5.23 (dd, 1H, J_{3′,F}=54.36 and J_{3′,4′}=2.02 Hz, H3′), 4.36-4.45 (m, 2H, H⁴′, H2′), 3.78(s, 6H, OMe), 3.40-3.55 (m, 2H, H5′, H5″).

### (ii) Preparation of 5′-O-(4,4′-dimethoxytrityl)-β-D-ribofuranosyl 3′-deoxy-3′-fluoro-uridinyl -2-O-phosphoramidite (VIIIa)

Compound 28 (100 mg) obtained from previous step was dissolved in dry DCM (1 ml) followed by the addition of diisopropyl ethyl amine (40 μl) and chloro (2-cyanoethoxy)-(N,N-diisopropylamino)-phosphine (40 [l) and the reaction mixture was stirred at room temperature for 2 h. The contents were then diluted with dry DCM and washed with 5% NaHCO₃ solution. The organic phase was dried over anhydrous Na₂SO₄ and concentrated to foam. The residue was dissolved in DCM and precipitated with hexane to obtain the title compound (105 mg).

Yield: 80%

³¹P NMR (CDCl₃) δ: 151.41, 152.51.

### EXAMPLE 6

### Process for preparing 6′-(O-Dimethoxytrityl) -hydroxymethyl-4′-hydroxy-3′ (purinyl/pyrimidinyl)-cyclohexene-2′-O-phosphoramidite (X)

Experimental Procedure for 24:

To a suspension of liquid ammonia (700 mL) and sodium (4.78 g, 207.8 mmol) vigorously stirring at −78 ° C. was added dropwise the solution of 23 (8 g, 25.97 mmol) in 10/1 mixture of THF (100 mL)/EtOH (10 mL) and the stirring was continued for 15 min. The reaction mixture was quenched with saturated aqueous NH₄Cl solution and was kept at room temperature overnight to allow liquid ammonia (B. P=−33° C.) to evaporate. THF/EtOH was removed on rota in vacuo and the residue was diluted with DCM and water was given. Aqueous layer was extracted 2 times with DCM and the combined extracts were washed with the brine solution. Organic layer was dried over Na₂SO₄, filtered, concentrated in vacuo and purified through column chromatography (Pet ether/AcOEt=85/15) to result 24 (3.4 g) in 78% yield as a pale yellow thick liquid.

¹H NMR (200 MHz, CDCl3): δ 6.52 (m, 1H), 6.10 (m, 1H), 4.85 (s, 1H), 4.56 (s, 1H), 3.26 (merged, 1H), 3.18 (s, 3H), 3.15 (s, 3H), 2.87 (m, 1H), 2.42 (m, 1H), 0.86 (dd, 1H, J=12.38, 2.27 Hz) ; ¹³C NMR (200 MHz, CDCl3): δ 137.92, 128.39, 119.24, 70.20, 51.66, 50.71, 49.66, 45.62, 36.21.

Experimental Procedure for 25:

To a solution of 24 (3 g, 17.6 mmol) in pyridine (15 mL) was added benzoyl chloride (2.7 mL, 19.41 mmol) and allowed to stir at rt for 4 h. Pyridine was removed in vacuo and the residue was diluted with AcOEt. Water wash and brine wash were given to the organic layer, dried over sodium sulphate, concentrated in vacuo. The residue was subjected to filtration column (Pet ether/AcOEt=89/11) to afford 25 (4.5 g) in 93% yield.

¹H NMR (200 MHz, CDCl3): δ 7.99 (m, 2H), 7.58 (m, 3H), 6.44 (m, 1H), 6.10 (m, 1H), 5.60 (m, 1H), 3.40 (m, 1H), 3.25 (s, 3H), 3.18 (s, 3H), 2.96 (m, 1H), 2.57 (m, 1H), 1.19 (dd, 1H, J=12.50, 2.24 Hz) ; ¹³C NMR (200 MHz, CDCl3): δ 166.36, 135.96, 132.77, 130.08, 129.41, 128.21, 118.92, 73.71, 51.80, 49.82, 48.62, 45.15, 33.39.

Experimental Procedure for 26:

Solution of 25 (4.5 g, 16.4 mmol) in 180 mL of 6/1 mixture of acetic acid/water was refluxed for 4 h. Solvent was removed in vacuo, residue diluted with the AcOEt and water wash, saturated aqueous NaHCO₃ wash and finally brine wash were given. The organic layer was dried over sodium sulphate, concentrated in vacuo. The residue subjected to column purification (Pet ether/AcOEt=90/10) to afford 26 in 81% yield.

¹H NMR (200 MHz, CDCl3): δ 7.98 (m, 2H), 7.59 (m, 3H), 6.80 (m, 1H), 6.49 (m, 1H), 5.66 (m, 1H), 3.54 (m, 1H), 3.07 (t, 1H, J=7.2, 3.54 Hz), 2.64 (m, 1H), 1.49 (dd, 1H, J=13.52, 3.03 Hz); ¹³C NMR (200 MHz, CDCl3): δ 201.01, 166.15, 134.45, 133.24, 129.55, 128.93, 128.43, 69.06, 51.88, 47.42, 32.55

Experimental Procedure for BV Xxidation:

MCPBA (77%, 1.25 g, 5.57 rnmol) was added to a stirred suspension of 26 (1.27 g, 5.57 mmol) and Na₂C0₃ (594 mg, 5.6 mmol) in DCM (40 mL) at 0. The reaction mixture was stirred for 6 h at rt before it was quenched with 10% aq. solution of Na₂S₂O₅ (15 mL). The organic layer was separated and aq. layer was extracted with DCM (2×30 mL). The combined organic extract was washed with saturated aq. NaHCO₃ solution (10 mL) followed by brine (30 mL), prior to drying over anhydrous Na₂S₂O₅. Filtration column was done for the unseparable regioisomeric mixture of lactones 27 and 28 (1.2 g, 92%) in 70:30 ratio. The mixture of lactones was subjected to LAH reduction.

LC MS for 27 and 28: calcd for C₁₄H₁₂O₄ (M+H)⁺: 244.07. Found: 267.26 (M+Na)⁺

Experimental Procedure for LAH Reduction:

To the mixture of 27 and 28 (790 mg, 3.23 mmol) in dry THF (140 mL), cooled at −15 ° C., was added LAH (370 mg, 9.71 mmol) and the reaction mixture stirred for 2 h at the same temperature. The reaction mixture was cautiously quenched with AcOEt followed by saturated Na₂S0₄ solution, to precipitate out aluminium salts. After filtration, the filtrate was concentrated in vacuo and subjected to column chromatography over silica gel to furnish inseparable mixture of 29 and 30 (330 mg, eluted in MeOH-AcOEt, 1:99) along with 31, epimeric product of 29 (65 mg, eluted in 100% ethyl acetate) in 8:2 ratio at 85% yield.

LC MS for 29 & 30: calcd for C₇H₁₂O₃ (M+H)⁺: 144.07. Found: 167.17 (M+Na)⁺

LC MS for 31: calcd for C₇H₁₂O₃ (M+H)⁺: 144.07. Found: 167.09 (M+Na)⁺

Experimental Procedure for 32:

To a solution of 29 and 30 (540 mg, 3.78 mmol) in dry 1,4-dioxane (10 mL) was added PTSA (30 mg, 0.19 mmol) and benzaldehydedimethyl acetal (0.73 mL, 4.91 mmol) and stirred for 24 h at rt. Ice was added to the reaction mixture and stirred for 0.5 h and was extracted with AcOEt three times. The combined organic layers were washed with water, brine solution and was dried over Na₂SO₄ prior to its concentration. The crude was subjected to column purification to furnish 565 mg of 32 (20% pet ether in ethyl acetate) in 64.5% yield along with the recovery of 30 (140 mg in 26% yield).

¹H NMR (200 MHz, CDCl₃): δ 7.34-7.54 (m, 5H), 5.8-5.89 (dm, 1H, J=9.78 Hz), 5.63 (s, 1H), 5.47 (dd, 1H, J=9.86, 1.72 Hz), 4.40 (m, 1H), 4.25 (dd, 1H, J=10.82, 4.53 Hz), 3.81-3.94 (m, 1H), 3.69 (t, 1H, J=11.42 Hz), 2.38-2.54 (m, 1H, OH), 2.15-2.23 (m, 1H), 1.86 (td, 1H, J=12.57, 4.98 Hz); ¹³C NMR (CDCl₃, 200 MHz): δ 138.18, 130.23, 128.99, 128.36, 126.19, 102.36, 75.35, 70.65, 65.22, 40.40, 37.25. LC MS: calcd for C₁₄H₁₆O₃ (M+H)⁺: 232.10. Found: 255.16 (M+Na)⁺

Experimental Procedure for 33:

To a solution of 30 (60 mg, 0.41 mmol) in 4 mL pyridine was added acetic anhydride (0.2 mL, 2 mmol) and stirred for overnight. Pyridine was evaporated on rota and the residue was diluted with DCM. To the organic layer water wash and brine wash were given and was dried over Na₂SO₄ prior to its concentration. Crude was subjected to column purification to furnish the triacetate 33 in (60 mg at 72% yield, eluted in 12% AcOEt in pet ether). The triacetate 33 was derived from minor regio-isomer in the Bayer-Villiger's oxidation, confirmed using ¹H-¹H COSY.

¹H NMR (200 MHz, CDCl₃): δ 5.86-5.92 (m, 1H), 5.71-5.79 (m, 1H), 5.19 (m, 1H), 5.05-5.13 (m, 1H), 4.05 (d, 1H, J=6.58 Hz), 2.64 (m, 1H), 2.08 (s, 3H), 2.06 (s, 3H), 2.05 (s, 3H), 1.91-2.02 (merged m, 1H), 1.69-1.82 (ddd, 1H, J=10.54, 7.37, 3.12 Hz).

LC MS: calcd for C₁₃H₁₈O₆ (M+H)⁺: 270.11 Found: 293.18 (M+Na)⁺

Although certain presently preferred embodiments of the invention have been specifically described herein, it will be apparent to those skilled in the art to which the invention pertains that variations and modifications of the various embodiments shown and described herein may be made without departing from the spirit and scope of the invention. Accordingly, it is intended that the invention be limited only to the extent required by the appended claims and the applicable rules of law.

We claim:

1. A 2′-O-phosphoramidite of N-type (3′-endo) and S-type (2′-endo) locked nucleosides of formula I, 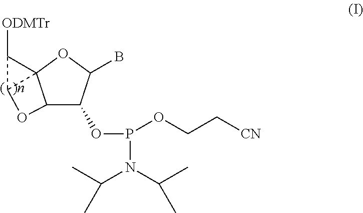
wherein the dotted lines in formula I represent 3′-O,5′-C or 3′-O,4′-C-oxyalkylene linkage respectively, and
wherein n=1, 2 or 3, and B is selected from a pyrimidine or a purine nucleic acid base.

2. A 2′-5′-linked ribo/deoxyribonucleic acid oligomers comprising 2′-O-phosphoramidite of N-type (3′-endo) or S-type (2′-endo) locked nucleosides of formula I 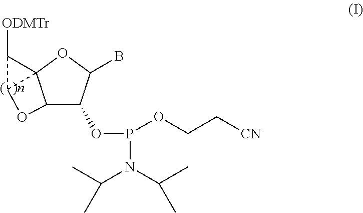
wherein the dotted lines in nucleosides of formula I represent 3′-O,5′-C or 3′-O,4′-C-oxyalkylene linkage respectively, and
wherein n=1, 2 or 3 and B is selected from a pyrimidine or a purine nucleic acid base.

3. A 2′-O-phosphoramidite of N-type locked nucleoside according to claim 1 comprising the composition of formula II 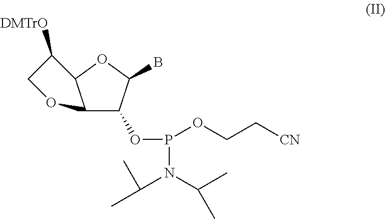
wherein B is a pyrimidine or purine nucleic acid base.

4. A 2′-O-phosphoramidite of N-type locked nucleoside according to claim 3, wherein B is a pyrimidine base.

5. A 2′-5′-linked ribo/deoxyribonucleic acid oligomers of Formula III comprising 2′-O-phosphoramidite of N-type (3′-endo) of formula II according to claim 3, 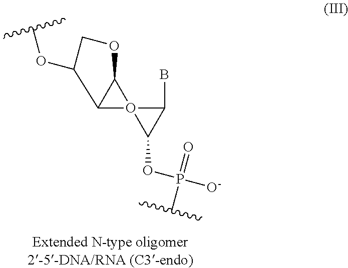
wherein B is pyrimidine or purine nucleic acid base.

6. A 2′-5′-linked ribo/deoxyribonucleic acid oligomers according to claim 5, wherein B is a pyrimidine base.

7. A 2′-5′-linked ribo/deoxyribonucleic acid oligomers according to claim 6 comprises a sequence selected from the group comprising of following sequences, wherein U^{N} is a N-type locked nucleoside monomer
5′-CAC CAT TGT CAC AC [symbol] CCA-2′
5′-CAC CAT TG [symbol] CAC AC [symbol] CCA-2′
5′-CAC CA [symbol] TG [symbol] CAC AC [symbol] CCA-2′
5′-CCT CTT ACC TCA GT [symbol] ACA-2′
5′-CCT CTT ACC [symbol] CA GT [symbol] ACA-2′
5′-CCT CT [symbol] ACC [symbol] CA GT [symbol] ACA-2′

8. A 2′-O-phosphoramidite of N-type locked nucleoside according to claim 3, prepared by a process which comprises the steps of:
(i) reacting 3,6-anhydro-5′-hydroxy-1,2-isopropylidene-Glucofuranose (5) with allyloxycarbonyl chloride to obtain 5′-O-allyloxycarbonyl-3,6-anhydro-1,2-isopropylidene-Glucofuranose (6) 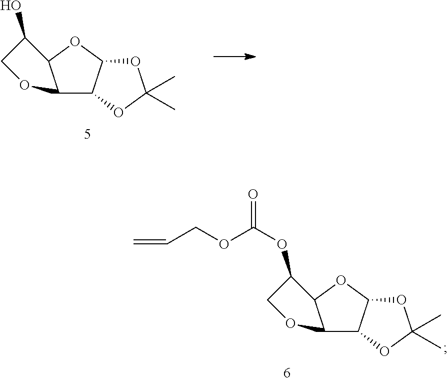
(ii) treating compound of formula (6) as obtained in step (i) with a mixture of acetic acid, acetic anhydride and sulfuric acid to give 1,2-di-O-acetyl-3,6-anhydro-5′-O-allyloxycarbonyl-α,β-Glucofuranose (7) 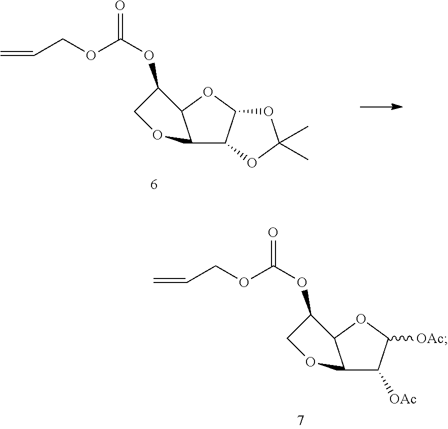
(iii) reacting compound of formula (7) as obtained in step (ii) with nucleobase in presence of N,O-Bis(trimethylsilyl)acetamide (BSA) and Trimethylsilyl Trifluoromethanesulfonate (TMSOTf) to obtain 2′-O-acetyl-3,6-anhydro-5′-O-allyloxycarbonyl-nucleoside (8) 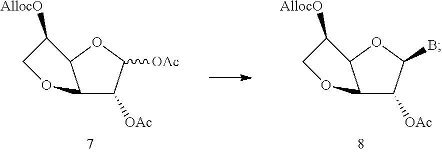
(ii) deprotecting allyloxycarbonyl group at 5′-position of compound (8) as obtained in step (iii) in presence of Tris(dibenzylidene acetone) dipalladium [Pd₂(dba)₃] catalyst to obtain 5′-hydroxy-2′-O-acetyl-3,6-anhydro-nucleoside (9) 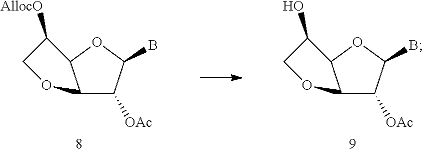
(iii) treating 5′-hydroxy-2′-O-acetyl-3,6-anhydro-nucleoside (9) as obtained in step (iv) with 4,4′-dimethoxytritylchloride to give 5′-O-dimethoxytrityl-2′-O-acetyl-3,6-anhydro-nucleoside (10) 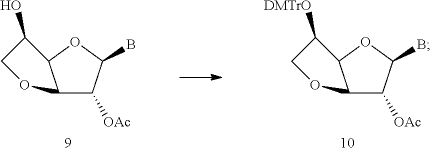
(iv) hydrolysing compound (10) as obtained in step (v) with aqueous ammonia in presence of methanol to yield 5′-O-dimethoxytrityl-2′-hydroxy-3,6-anhydro-nucleoside (11) 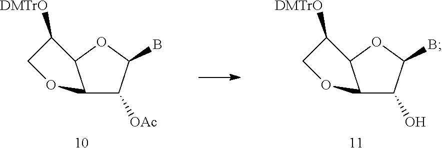
and
(v) treating compound (11) as obtained in step (vi) with chloro-(2-cyanoethoxy)-N,N-diisopropyl amino)-phosphine in presence of Diisopropylethylamine (DIPEA) to give final compound 5′-O-Dimethoxytrityl-3′-O,5′-C-methylene(uridine)xylonucleoside-2′-O-phosphoramidite (II)

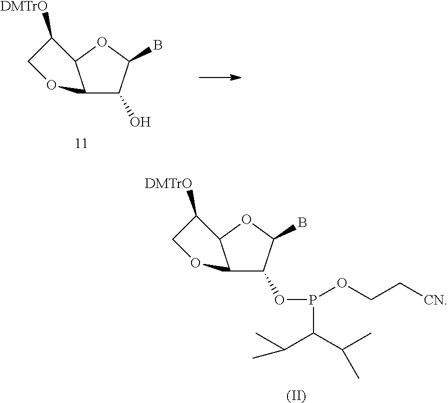

9. A 2′-O-phosphoramidite of S-type locked nucleoside according to claim 1 comprising the composition of formula IV 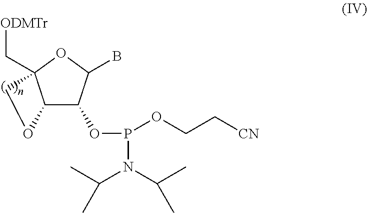
wherein B is a pyrimidine nucleic acid base and n=1, 2, 3.

10. A 2′-O-phosphoramidite of S-type locked nucleoside according to claim 9, wherein B is uracil.

11. A 2′-5′-linked ribo/deoxyribonucleic acid oligomers of formula V, comprising a 2′-O-phosphoramidite S-type (2′-endo) locked nucleosides according to claim 9 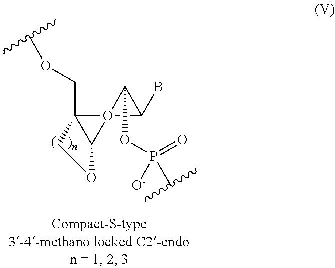
wherein B is pyrimidine nucleic acid base.

12. A 2′-5′-linked ribo/deoxyribonucleic acid oligomers according to claim 11, wherein B is uracil.

13. A 2′-5′-linked ribo/deoxyribonucleic acid oligomers according to claim 11 comprises a sequence selected from the group comprising of following sequences, wherein U^{S} is S-type locked nucleoside monomer
5′-CAC CAT TGT CAC AC [symbol] CCA-2′
5′-CAC CAT TG [symbol] CAC AC [symbol] CCA-2′
5′-CAC CA [symbol] TG [symbol] CAC AC [symbol] CCA-2′
5′-CCT CTT ACC TCA GT [symbol] ACA-2′
5′-CCT CTT ACC [symbol] CA GT [symbol] ACA-2′
5′-CCT CT [symbol] ACC [symbol] CA GT [symbol] ACA-2′

14. A 2′-O-phosphoramidite of S-type locked nucleoside according to claim 9, prepared by a process comprising the steps of:
(A) protecting 2′,3′ position of uridine by cyclohexylidene group to give 2% 3′-O-cyclohexylidene-uridine, followed by oxidizing 5′-OH group of 2′,3′-protected nucleoside to give 5′-aldehydo-2′,3′-O-cyclohexylidene-nucleoside (13) 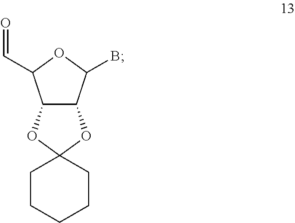
B) hydroformylating C-4′position and reducing 5′-aldehyde group of compound (13) as obtained in step (A) to give 4′-hydroxymethyl-2′,3′-O-cyclohexylidene-nucleoside (14) 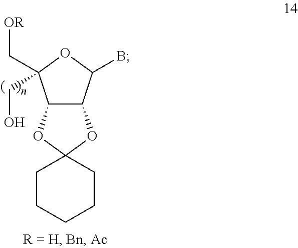
C) tosylating 4′-position; deprotecting 2′,3′ position to obtain 4′-p-toluenesulphonyl-methyl-uridine and reacting 4′-p-toluenesulphonyl-methyl-uridine with optimize amount of 4,4′-dimethoxytritylchloride, followed by ring closure to yield 5′-O-DM Tr-O3′-4′-methylene bridged uridine (15) 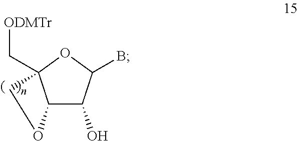
and
D) converting compound (15) as obtained in step (C) to 2′-O-phosphoramidite of S-type locked nucleoside (IV), wherein, n=1 and B═U

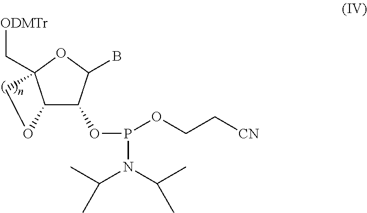

15. A process for preparing 2′-O-phosphoramidite of S-type locked nucleoside according to claim 9, the process comprising the steps of
preparing O3′-4′-methylene-bridged uridine from 4′-p-Toluenesulphonylmethyl-uridine;
reacting the O3′-4′-methylene-bridged uridine with DMTr to yield 5′-O-DMTr-O3′-4′-methylene bridged uridine (15); and
converting compound (15) to 2′-O-phosphoramidite of S-type locked nucleoside (IV), wherein, n=1 and B═U.

16. A 2′-O-phosphoramidite of unlocked nucleoside of formula (VI) 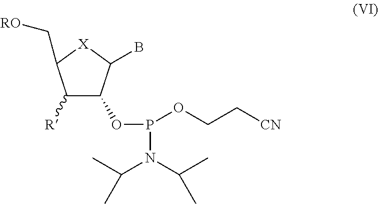
wherein R=DMTr; R′═F or H; X=O or (C═C) and B=pyrimidine or purine nucleic acid base.

17. A 2′-O-phosphoramidite of formula VI according to claim 16, wherein B is a pyrimidine base.

18. A 2′-5′-linked nucleic acid oligomer of Formula VII 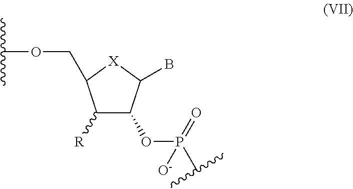
wherein R′═F or H; X═O or (C═C) and B is pyrimidine or purine nucleic acid base.

19. A 2′-5′-linked nucleic acid oligomer according to claim 18, wherein B is a pyrimidine base.

20. A 2′-O-phosphoramidite of formula VI according to claim 16, wherein the compound is 3′-fluoro-3′-deoxy-2′-O-phosphoramidite of formula VIII 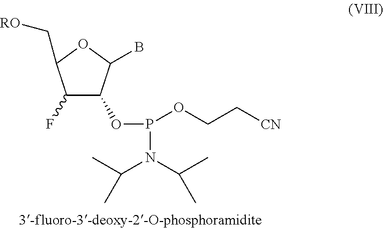
wherein B is a pyrimidine or purine nucleic acid base, and R is dimethoxyltrityl group (DMTr).

21. A 2′-O-phosphoramidite of formula VI according to claim 20, wherein said 3′-fluoro-3′-deoxy-2′-O-phosphoramidite of formula VIII is selected from VIII a and VIII b,

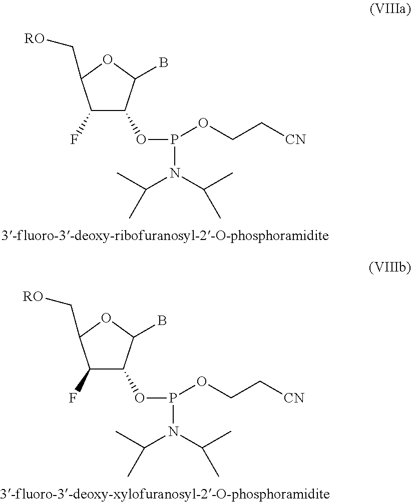

22. A 2′-5′-linked nucleic acid oligomer according to claim 18, wherein the compound is 3′-fluoro-2′-5′-linked nucleic acid oligomer of formula IX, 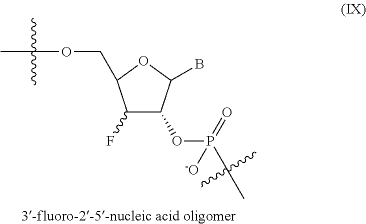
wherein B is a pyrimidine or purine nucleic acid base.

23. A 2′-5′-linked nucleic acid oligomer according to claim 22, wherein said 3′-fluoro-2′-5′-linked nucleic acid oligomer of formula IX is selected from 3′-fluoro-2′-5′-linked ribo/deoxy-ribonucleic acid (2′-5′-RNA/DNA) of formula (IXa) and xylo/deoxy-xylonucleic acid (2′-5′-XNA/dXNA) of formula (IXb), 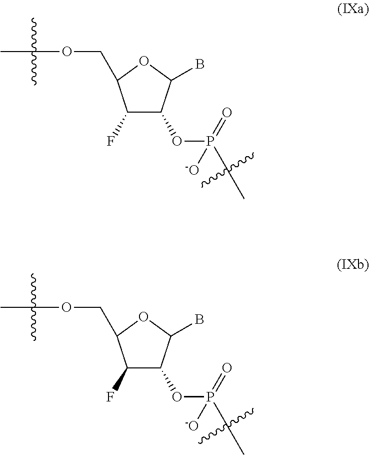
wherein B is a pyrimidine or purine nucleic acid base.

24. A 3′-fluoro-2′-5′-linked ribo/deoxy-ribonucleic acid (2′-5′-RNA/DNA) oligomer according to claim 23 comprises a sequence selected from the group consisting of following sequences
5′-CAC CAT TGT CAC AC [symbol] CCA-2′
and
5′-CAC CAT TG [symbol] CAC AC [symbol] CCA-2′,
wherein U^{rF} is fluoro ribonucleoside monomer.

25. A 3′-fluoro-2′-5′-linked xylo/deoxy-xylonucleic acid (2′-5′-XNA/dXNA) oligomer according to claim 23 comprises a sequence selected from the group consisting of following sequences
5′-CAC CAT TGT CAC AC [symbol] CCA-2′
and
5′-CAC CAT TG [symbol] CAC AC [symbol] CCA-2′,
wherein U^{xF} is fluoro xylonucleoside monomer.

26. A 3′-fluoro-3′-deoxy-xylofuranosyl-2′-O-phosphoramidite according to claim 21, prepared by a process comprising the steps of:
(I) converting 1, 2 and 5, 6 isopropylidine protected glucose to 3′-epimer (17) through oxidation and reduction steps 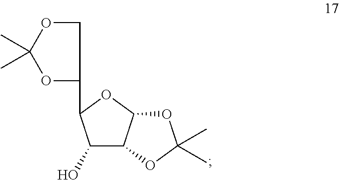
(II) tosylating at the 3′-position of compound 17 to give compound (18) 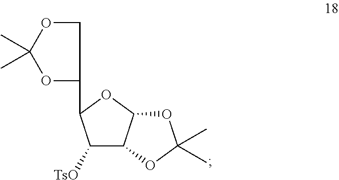
(III) displacing tosyl group of compound (18) as obtained in step II with fluoride anion to give C3′-fluoro intermediate (19) 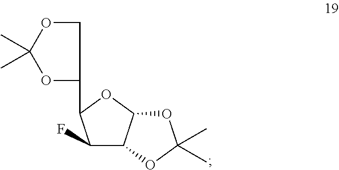
and
(IV) converting fluoro intermediate (19) as obtained in step III to 3′-fluoro-3′-deoxy-xylofuranosyl 2′-O-phosphoramidite (VIIIb)

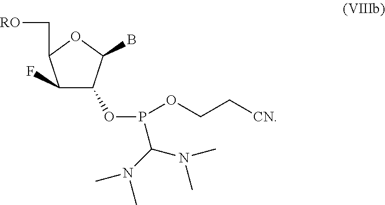

27. A 3′-fluoro-3′-deoxy-ribofuranosyl-2′-O-phosphoramidite according to claim 21, prepared by a process comprising the steps of:
(V) reacting 3′-deoxy-3′-fluoro-5′-hydroxy ribofuranosyl uridine (20) 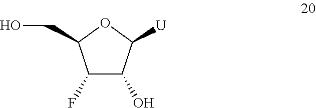
with 4,4′-Dimethoxy tritylchloride, in the presence of catalytic amount of 4-dimethylaminopyridine dissolved in pyridine to obtain 5′-O-dimethoxytrityl-3′-deoxy-3′-fluoro-ribofuranosyl uridine (21) 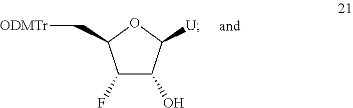
(VI) dissolving compound (21) as obtained in step (V) in dry DCM followed by the addition of diisopropyl ethyl amine and chloro (2-cyanoethoxy) (N,N-diisopropylamino)-phosphine to obtain 3′-fluoro-3′-deoxy-ribofuranosyl-2′-O-phosphoramidite (VIIIa)

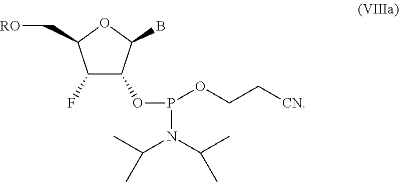

28. A 2′-O-phosphoramidite of formula VI according to claim 16, wherein the compound is 6′-O-Dimethoxytrityl)-hydroxymethyl-4′-hydroxy-3′-(purinyl/pyrimidinyl)-cyclohexene-2′-O-phosphoramidite of formula X 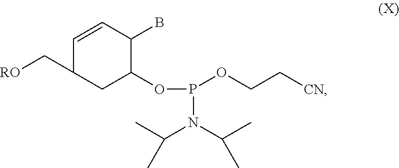
wherein R=DMTr and B is a pyrimidine or purine nucleic acid base.

29. A 2′-5′-linked nucleic acid oligomer according to claim 18, wherein R′═H; X═(C═C) and wherein B is a pyrimidine or purine nucleic acid base.

30. A 6′-(O-Dimethoxytrityl) -hydroxymethyl-4′-hydroxy-3′ (purine/pyrimidinyl)-cyclohexene-2′-O-phosphoramidite according to claim 28, prepared by a process comprising the steps of:
(1) hydrolyzing racemic Diels-Alder adduct of formula 22 enantioselectively in phosphate buffer with a lipase (Candida cylindracea) 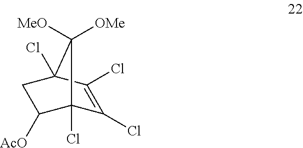
to yield a single enantiomer of formula 23 and leaving the acetate of formula 22 enriched in the other enantiomer (>95% based on NMR) 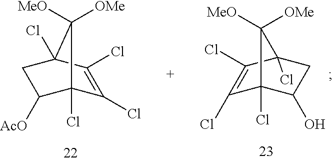
(2) treating alcohol of formula 23 as obtained in step (1) with a mixture of ammonia and sodium in THF/EtOH yielded product 24 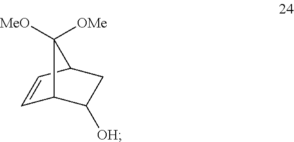
(3) treating compound of formula 24 as obtained in step (2) with benzoyl chloride to give 25; 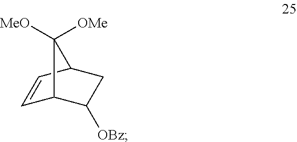
(4) ketal hydrolysis of 25 as obtained in step (3) to yield lactone 26 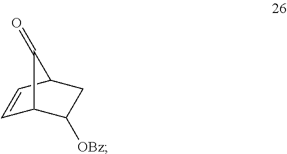
(5) oxidizing lactone 26 as obtained in step (4) with mCPBA to yield an inseparable mixture of 27 and 28 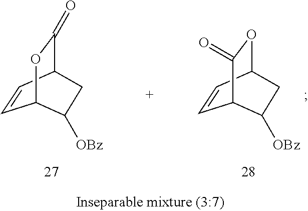
(6) reducing the above mixture (27 and 28) as obtained in step (5) to yield an inseparable mixture of 29 and 30 along with product 31 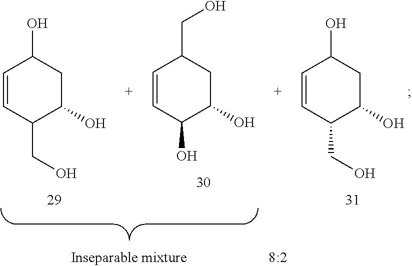
(7) treating mixture of 29 and 30 as obtained in step (6) with benzaldehydedimethylacetal to yield the benzylidene derivative 32 from 29, leaving triol 30 unreacted 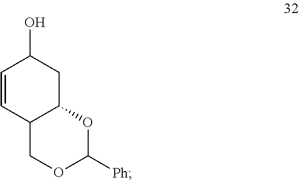
and
(8) converting triol 30 as obtained in step (7) to its triacetate derivative 33 for characterization 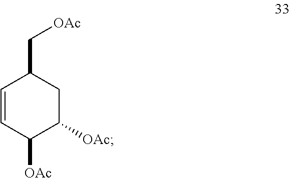
and
(9) converting stereochemically pure 30 as obtained in step (7) to the corresponding DMTr-protected base-containing phosphoramidite X

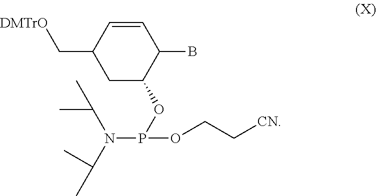

31. A composition comprising the phosphoramidite monomer units of claim 1 incorporated in 2′-5′-linked ribo/deoxyribonucleic acid oligomers and converted to duplexes with 3′-5′RNA.

32. A compositiong comprising the phosphoramidite monomer units according to claim 16 incorporated in 2′-5′-linked ribo/deoxyribonucleic acid oligomers and converted to duplexes with 3′-5′RNA.

33. A 2′-5′-linked nucleic acid oligomer according to claim 2, comprising 10-30 nucleoside units having 1 or more locked nucleoside monomer units.

34. A 2′-5′-linked nucleic acid oligomer according to claim 18, comprising 10-30 nucleoside units having 1 or more phosphoramidite nucleoside monomer units.


SEQUENCE LISTING (16 sequences)

SEQ ID NO: 1 - DNA, 18 bp, Artificial Sequence
  Synthesized in laboratory
caccattgtcacacncca

SEQ ID NO: 2 - DNA, 18 bp, Artificial Sequence
  Synthesized in laboratory
caccattgncacacncca

SEQ ID NO: 3 - DNA, 18 bp, Artificial Sequence
  Synthesized in laboratory
caccantgncacacncca

SEQ ID NO: 4 - DNA, 18 bp, Artificial Sequence
  Synthesized in laboratory
cctcttacctcagtnaca

SEQ ID NO: 5 - DNA, 18 bp, Artificial Sequence
  Synthesized in laboratory
cctcttaccncagtnaca

SEQ ID NO: 6 - DNA, 18 bp, Artificial Sequence
  Synthesized in laboratory
cctctnaccncagtnaca

SEQ ID NO: 7 - DNA, 18 bp, Artificial Sequence
  Synthesized in laboratory
caccattgtcacactcca

SEQ ID NO: 8 - DNA, 18 bp, Artificial Sequence
  Synthesized in laboratory
cctcttacctcagttaca

SEQ ID NO: 9 - DNA, 18 bp, Artificial Sequence
  Synthesized in laboratory
caccattgtcacactcca

SEQ ID NO: 10 - DNA, 18 bp, Artificial Sequence
  Synthesized in laboratory
cctcttacctcagttaca

SEQ ID NO: 11 - DNA, 18 bp, Artificial Sequence
  Synthesized in laboratory
tggagtgtgacaatggtg

SEQ ID NO: 12 - DNA, 18 bp, Artificial Sequence
  Synthesized in laboratory
uggagugugacaauggug

SEQ ID NO: 13 - DNA, 18 bp, Artificial Sequence
  Synthesized in laboratory
tgtaactgaggtaagagg

SEQ ID NO: 14 - DNA, 18 bp, Artificial Sequence
  Synthesized in laboratory
uguaacugagguaagagg

SEQ ID NO: 15 - DNA, 18 bp, Artificial Sequence
  Synthesized in laboratory
tggagtgtgacaatggtg

SEQ ID NO: 16 - DNA, 18 bp, Artificial Sequence
  Synthesized in laboratory
uggagugugacaauggug